(19) United States
(12) Patent Application Publication
(10) Pub. No.: US 2023/0133684 A1
(43) Pub. Date: May 4, 2023

(54) HEATING ELEMENT

(71) Applicant: Nicoventures Trading Limited, London (GB)

(72) Inventors: Howard Rothwell, London (GB); Tom Woodman, London (GB); Sam Lai, London (GB)

(21) Appl. No.: 17/907,506

(22) PCT Filed: Mar. 26, 2021
(86) PCT No.: PCT/GB2021/050760

§ 371 Date: Sep. 27, 2022

(30) Foreign Application Priority Data
Mar. 31, 2020 (GB) 2004702.3

(51) Int. Cl. A61M 15/06; A24F 40/10

(52) U.S. Cl. CPC A61M 15/06; A24F 40/10; A61M 11/042

(57) ABSTRACT

The present disclosure relates to an article for use with an electrically operated non-combustible aerosol delivery system, the article including a generally planar aerosol generating component suspended within an aerosol generating chamber, the chamber having one or more air inlets and one or more outlets defining a flow path therebetween, wherein during activation of the aerosol generating component a first temperature profile having a negative gradient is established along a portion of the flow path from inlet to outlet.

### PRIORITY CLAIM

The present application is a National Phase entry of PCT Application No. PCT/GB2021/050760, filed Mar. 26, 2021, which claims priority from GB Application No. 2004702.3, filed Mar. 31, 2020, each of which is hereby fully incorporated herein by reference.

### TECHNICAL FIELD

The present disclosure relates to a delivery system, in particular to a non-combustible aerosol delivery system and to components of said aerosol delivery system. The present disclosure further relates to methods of generating and delivering an aerosol using the non-combustible aerosol delivery system and components disclosed herein.

### BACKGROUND

Non-combustible aerosol delivery systems which generate an aerosol for inhalation by a user are known in the art. Such systems typically comprise an aerosol generator which is capable of converting an aerosolizable material into an aerosol. In some instances, the aerosol generated is a condensation aerosol whereby an aerosolizable material is first vaporized and then allowed to condense into an aerosol. In other instances, the aerosol generated is an aerosol which results from the atomization of the aerosolizable material. Such atomization may be brought about mechanically, e.g. by subjecting the aerosolizable material to vibrations so as to form small particles of material that are entrained in airflow. Alternatively, such atomization may be brought about electrostatically, or in other ways, such as by using pressure etc.

Since such aerosol delivery systems are intended to generate an aerosol which is to be inhaled by a user, consideration should be given to the characteristics of the aerosol produced. These characteristics can include the size of the particles of the aerosol, the total amount of the aerosol produced, etc.

Where the aerosol delivery system is used to simulate a smoking experience, e.g. as an e-cigarette or similar product, control of these various characteristics is especially important since the user may expect a specific sensorial experience to result from the use of the system.

It would be desirable to provide aerosol delivery systems which have improved control of these characteristics.

### SUMMARY

In one aspect there is provided an article for use with an electrically operated non-combustible aerosol delivery system, the article comprising a generally planar aerosol generating component suspended within an aerosol generating chamber, the chamber having one or more air inlets and one or more outlets defining a flow path therebetween, wherein during activation of the aerosol generating component a first temperature profile having a negative gradient is established along a portion of the flow path from inlet to outlet.

The longitudinal axis of the aerosol generating component may be substantially parallel to the flow path.

The negative temperature gradient may be established along less than 50% of the flow path. The negative temperature gradient may be established along less than 20% of the flow path.

The negative temperature gradient may be established along the first 5% of the flow path.

The negative temperature gradient is established from a peak temperature of above 250° C.

The peak temperature may be established in proximity to the one or more air inlet(s) of the aerosol generating chamber. The peak temperature may be established within 20%, of the opening of the air inlet(s) into the aerosol generating chamber.

The aerosol generating component may be configured to dissipate greater energy at an upstream location relative to a downstream location.

The aerosol generating component may be configured with a capillary structure at an upstream portion which has a capillarity inferior in terms of feed rate compared to a capillary structure at a corresponding downstream portion. An upstream portion of the aerosol generating component may be configured to have a relatively greater electrical resistance than a corresponding downstream portion.

The aerosol generating component may comprise one or more apertures or slots extending from one surface of the planar aerosol generating component to the opposite surface.

The one or more slots may originate from the periphery of the aerosol generating component.

The one or more slots may be arranged perpendicularly to the longitudinal extent of the aerosol generating component such that a plurality of portions extending perpendicularly to the longitudinal extent of the aerosol generating component are formed. The width of an upstream portion may be less than the width of a subsequent downstream portion. The ratio of the width of an upstream portion to a subsequent downstream portion may be less than 1, such as less than 0.9, less than 0.8, less than 0.7, or less than 0.6, but not less than 0.5.

A second temperature profile may be established along a subsequent portion of the flow path. The second temperature profile may have a negative gradient which is smaller than the gradient of the first temperature profile. The second temperature profile may have a negative gradient which is greater than gradient of the first temperature profile.

In a further aspect, there is provided an aerosol generating component for use with an electrically operated non-combustible aerosol delivery system, the aerosol generating component defining a longitudinal-axis and being configured to be heated heterogeneously along its longitudinal axis. During activation of the aerosol generating component, a first temperature profile with a negative gradient may be established along a portion of the longitudinal axis of the aerosol generating component.

The negative gradient may be established along less than 50% of the longitudinal axis of the aerosol generating component.

The negative gradient may be established along less than 20% of the longitudinal axis of the aerosol generating component.

The negative gradient may be established along the first 5% of the longitudinal axis of the aerosol generating component.

The aerosol generating component may be formed from a woven or weave structure, mesh structure, fabric structure, open-pored fiber structure, open-pored sintered structure, open-pored foam or open-pored deposition structure.

There is also provided a non-combustible aerosol provision system comprising the article as defined herein and a device comprising a power source and a control unit.

There is also provided a non-combustible aerosol provision system comprising the aerosol generating component as defined herein and a device comprising a power source and a control unit.

### BRIEF DESCRIPTION OF THE DRAWINGS

Various embodiments will now be described in detail by way of example only with reference to the accompanying drawings in which:

FIG. 1 provides a schematic overview of certain components of a non-combustible aerosol delivery system as described herein.

FIG. 2 provides an exploded view of an atomizer and associated components which according to various aspects of the present disclosure.

FIG. 3 provides a view of certain components of FIG. 2 in a stage of assembly.

FIG. 4 provides a view of certain components of FIG. 2 in a further stage of assembly relative to that shown in FIG. 3.

FIG. 5 provides a view of certain components of FIG. 2 in a further stage of assembly relative to that shown in FIG. 4.

FIG. 6 provides a view of certain components of FIG. 2 in a further stage of assembly relative to that shown in FIG. 5.

FIG. 7 provides a schematic cross section parallel to the longitudinal axis though the atomizer depicted in FIGS. 2 to 6.

FIG. 8 provides a perspective view of an exemplary aerosol generating component according to the present disclosure.

FIG. 8a provides a schematic illustration of aerosolizable material being fed to the periphery of an aerosol generating component in a plane parallel to the aerosol generating component.

FIG. 8b provides a schematic illustration of aerosolizable material being fed to the periphery of an aerosol generating component in a plane perpendicular to the aerosol generating component.

FIG. 8c provides an exploded view of capillary frame elements and aerosol generating component according to the present disclosure.

FIG. 9a provides a perspective view of a schematic illustration of an aerosol generating component held within a capillary frame according to the present disclosure.

FIG. 9b provides a schematic cross section perpendicular to the longitudinal axis of an aerosol generating chamber of an aerosol generating component held within a capillary frame according to the present disclosure.

FIG. 9c provides a schematic cross-section of a capillary frame having an aerofoil edge profile.

FIGS. 9d to 9f provide images of exemplary aerosol generating components according to the present disclosure.

FIGS. 10a and 10b provide schematic cross sectional views parallel to the longitudinal axis of a reservoir of an article according to the present disclosure.

FIG. 10c provides a schematic cross sectional plan perpendicular to the longitudinal axis of a reservoir of an article according to the present disclosure.

FIG. 11a provides a perspective image of an exemplary aerosol generating component and capillary gap according to the present disclosure.

FIG. 11b provides a perspective image of another exemplary aerosol generating component according to the present disclosure.

FIG. 12 provides a schematic plan view of an exemplary aerosol generating component projecting into a capillary gap according to the present disclosure.

FIG. 13 provides a schematic illustration of an aerosol generating chamber comprising an aerosol generating component.

FIG. 13a provides a schematic illustration of an aerosol generating chamber comprising an aerosol generating component suspended therein.

FIG. 13b provides a schematic plan view of an aerosol generating component wherein respective areas of different vaporization efficiency are depicted.

FIGS. 14a and 14b provide schematic illustrations of an aerosol generating chamber comprising an aerosol generating component, with the aerosol generating chamber having one or more air inlets in accordance with the present disclosure.

FIG. 15a provides an end view of an air inlet configuration of an aerosol generating chamber according to the present disclosure.

FIGS. 15b and 15c show air inlet configurations in accordance with the aerosol generating chamber shown in FIGS. 14a and 14b respectively.

FIG. 15d provides a graph showing the effect of varying the air inlet configuration on the particle size of an aerosol generated by an aerosol generating component as described herein.

FIGS. 16a to 16f show various air inlet configurations according to the present disclosure.

FIG. 17a provides a cross-sectional view parallel to the longitudinal axis of an article according to the present disclosure.

FIG. 17b provides a cross-sectional view of the article of FIG. 17a, the cross-section being taken in FIG. 17b perpendicular to the longitudinal axis of the article.

FIGS. 18a and 18b provide a graphic illustration of different temperature profiles along a flow path between an air inlet and an air outlet of an aerosol generating chamber as described herein.

FIG. 18c provides a schematic image of an aerosol generating component according to the present disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

As described above, the present disclosure relates to (but is not limited to) non-combustible aerosol provision systems and devices that release compounds from an aerosol-generating material (or aerosolizable material) without combusting the aerosol-generating material. Examples of such systems include electronic cigarettes, tobacco heating systems, and hybrid systems (which generate aerosol using a combination of aerosol-generating materials). In some examples, the non-combustible aerosol provision system is an electronic cigarette, also known as a vaping device or electronic nicotine delivery system (END), although it is noted that the presence of nicotine in the aerosol-generating material is not a requirement. In some examples, the non-combustible aerosol provision system is an aerosol-generating material heating system, also known as a heat-not-burn system. An example of such a system is a tobacco heating system. In some examples, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some examples, the hybrid system comprises a liquid or gel aerosol-generating material and a solid aerosol-generating material. The solid aerosol-generating material may comprise, for example, tobacco or a non-tobacco product.

Throughout the following description the terms “e-cigarette” and “electronic cigarette” may sometimes be used; however, it will be appreciated these terms may be used interchangeably with non-combustible aerosol (vapor) provision system or device as explained above.

In some examples, the present disclosure relates to consumables for holding aerosol-generating material, and which are configured to be used with non-combustible aerosol provision devices. These consumables are sometimes referred to as articles throughout the present disclosure.

The non-combustible aerosol provision system typically comprises a device part and a consumable/article part. The device part typically comprises a power source and a controller. The power source is typically an electric power source.

In some examples, the non-combustible aerosol provision system may comprise an area for receiving the consumable, an aerosol generator, an aerosol generation area (which may be within the consumable/article), a housing, a mouthpiece, a filter and/or an aerosol-modifying agent.

In some examples, the consumable/article for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

The systems described herein typically generate an inhalable aerosol by vaporization of an aerosol generating material. The aerosol generating material may comprise one or more active constituents, one or more flavors, one or more aerosol-former materials, and/or one or more other functional materials.

Aerosol-generating material may, for example, be in the form of a solid, liquid or gel which may or may not contain an active substance and/or flavorants. In some examples, the aerosol-generating material may comprise an “amorphous solid”, which may alternatively be referred to as a “monolithic solid” (i.e. non-fibrous). In some examples, the amorphous solid may be a dried gel. The amorphous solid is a solid material that may retain some fluid, such as liquid, within it. In some examples, the aerosol-generating material may for example comprise from about 50 wt%, 60 wt% or 70 wt% of amorphous solid, to about 90 wt%, 95 wt% or 100 wt% of amorphous solid.

The term “active substance” as used herein may relate to a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

The aerosol-former material may comprise one or more constituents capable of forming an aerosol. In some examples, the aerosol-former material may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials may comprise one or more of pH regulators, coloring agents, preservatives, binders, fillers, stabilizers, and/or antioxidants.

As used herein, the term “component” is used to refer to a part, section, unit, module, assembly or similar of an electronic cigarette or similar device that incorporates several smaller parts or elements, possibly within an exterior housing or wall. An electronic cigarette may be formed or built from one or more such components, and the components may be removably or separably connectable to one another, or may be permanently joined together during manufacture to define the whole electronic cigarette. The present disclosure is applicable to (but not limited to) systems comprising two components separably connectable to one another and configured, for example, as a consumable/article component capable of holding an aerosol generating material (also referred to herein as a cartridge, cartomizer or consumable), and a device/control unit having a battery for providing electrical power to operate an element for generating vapor from the aerosol generating material.

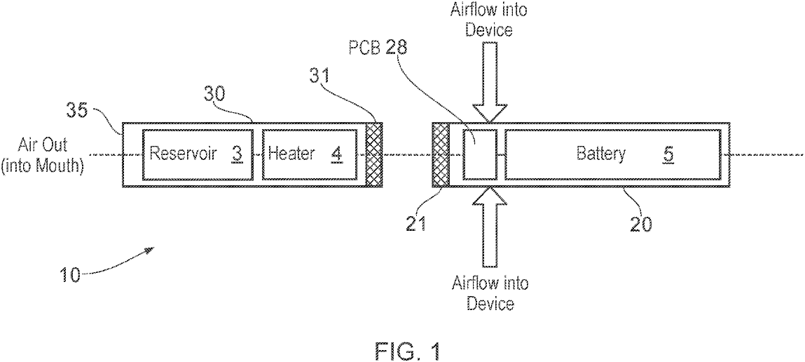

FIG. 1 is a highly schematic diagram (not to scale) of an example aerosol/vapor provision system such as an e-cigarette 10. The e-cigarette 10 has a generally cylindrical shape, extending along a longitudinal axis indicated by a dashed line, and comprises two main components, namely a control or power component or section 20 and a cartridge assembly or section 30 (sometimes referred to as a cartomizer, or clearomizer) that operates as a vapor generating component.

The cartridge assembly 30 includes a reservoir 3 containing an aerosolizable material comprising a liquid formulation from which an aerosol is to be generated, for example containing nicotine. As an example, the aerosolizable material may comprise around 1 to 3% nicotine and 50% glycerol, with the remainder comprising roughly equal measures of water and propylene glycol, and possibly also comprising other components, such as flavorings. The reservoir 3 has the form of a storage tank, being a container or receptacle in which aerosolizable material can be stored such that the aerosolizable material is free to move and flow within the confines of the tank. Alternatively, the reservoir 3 may contain a quantity of absorbent material such as cotton wadding or glass fibre which holds the aerosolizable material within a porous structure. The reservoir 3 may be sealed after filling during manufacture so as to be disposable after the aerosolizable material is consumed, or may have an inlet port or other opening through which new aerosolizable material can be added. The cartridge assembly 30 also comprises an electrical aerosol generating component 4 located externally of the reservoir tank 3 for generating the aerosol by vaporization of the aerosolizable material. In many devices, the aerosol generating component may be a heating element (heater) which is heated by the passage of electrical current (via resistive or inductive heating) to raise the temperature of the aerosolizable material until it evaporates. A liquid conduit arrangement such as a wick or other porous element (not shown) may be provided to deliver aerosolizable material from the reservoir 3 to the aerosol generating component 4. The wick has one or more parts located inside the reservoir 3 so as to be able to absorb aerosolizable material and transfer it by wicking or capillary action to other parts of the wick that are in contact with the vapor generating element 4. This aerosolizable material is thereby vaporized, to be replaced by new aerosolizable material transferred to the vapor generating element 4 by the wick.

A heater and wick combination, or other arrangement of parts that perform the same functions, is sometimes referred to as an atomizer or atomizer assembly, and the reservoir with its aerosolizable material plus the atomizer may be collectively referred to as an aerosol source. Various designs are possible, in which the parts may be differently arranged compared to the highly schematic representation of FIG. 1. For example, the wick may be an entirely separate element from the aerosol generating component, or the aerosol generating component may be configured to be porous and able to perform the wicking function directly (a metallic mesh, for example).

Arrangements of this latter type, where the functions of the vapor generation and wicking are combined in a single element, are discussed further below. In some cases, the conduit for delivering liquid for vapor generation may be formed at least in part from one or more slots, tubes or channels between the reservoir and the aerosol generating component which are narrow enough to support capillary action to draw source liquid out of the reservoir and deliver it for vaporization. In general, an atomizer can be considered to be an aerosol generating component able to generate vapor from aerosolizable material delivered to it, and a liquid conduit (pathway) able to deliver or transport liquid from a reservoir or similar liquid store to the aerosol generating component by a capillary force.

Typically, the aerosol generating component is located within an aerosol generating chamber that forms part of an airflow channel through the electronic cigarette/system. Vapor produced by the aerosol generating component is driven off into this volume, and as air passes through the volume, flowing over and around the vapor generating element, it collects the vapor whereby it condenses to form the required aerosol. The volume can be designated as a aerosol generating chamber.

Returning to FIG. 1, the cartridge assembly 30 also includes a mouthpiece 35 having an opening or air outlet through which a user may inhale the aerosol generated by the aerosol generating component 4, and delivered through the airflow channel.

The power component 20 includes a cell or battery 5 (referred to herein after as a battery, and which may be re-chargeable) to provide power for electrical components of the e-cigarette 10, in particular the aerosol generating component 4. Additionally, there is a printed circuit board 28 and/or other electronics or circuitry for generally controlling the e-cigarette. The control electronics/circuitry connect the vapor generating element 4 to the battery 5 when vapor is required, for example in response to a signal from an air pressure sensor or air flow sensor (not shown) that detects an inhalation on the system 10 during which air enters through one or more air inlets 26 in the wall of the power component 20 to flow along the airflow channel. When the aerosol generating component 4 receives power from the battery 5, the aerosol generating component 4 vaporizes aerosolizable material delivered from the reservoir 3 to generate the aerosol, and this is then inhaled by a user through the opening in the mouthpiece 35. The aerosol is carried from the aerosol source to the mouthpiece 35 along the airflow channel (not shown) that connects the air inlet 26 to the aerosol source to the air outlet when a user inhales on the mouthpiece 35. An airflow path through the electronic cigarette is hence defined, between the air inlet(s) (which may or may not be in the power component) to the atomizer and on to the air outlet at the mouthpiece. In use, the air flow direction along this airflow path is from the air inlet to the air outlet, so that the atomizer can be described as lying downstream of the air inlet and upstream of the air outlet.

In this particular example, the power section 20 and the cartridge assembly 30 are separate parts detachable from one another by separation in a direction parallel to the longitudinal axis, as indicated by the solid arrows in FIG. 1. The components 20, 30 are joined together when the device 10 is in use by cooperating engagement elements 21, 31 (for example, a screw, magnetic or bayonet fitting) which provide mechanical and electrical connectivity between the power section 20 and the cartridge assembly 30. This is merely an example arrangement, however, and the various components may be differently distributed between the power section 20 and the cartridge assembly section 30, and other components and elements may be included. The two sections may connect together end-to-end in a longitudinal configuration as in FIG. 1, or in a different configuration such as a parallel, side-by-side arrangement. The system may or may not be generally cylindrical and/or have a generally longitudinal shape. Either or both sections may be intended to be disposed of and replaced when exhausted (the reservoir is empty or the battery is flat, for example), or be intended for multiple uses enabled by actions such as refilling the reservoir, recharging the battery, or replacing the atomizer. Alternatively, the e-cigarette 10 may be a unitary device (disposable or refillable/rechargeable) that cannot be separated into two or more parts, in which case all components are comprised within a single body or housing. Embodiments and examples of the present invention are applicable to any of these configurations and other configurations of which the skilled person will be aware.

As mentioned, a type of aerosol generating component, such as a heating element, that may be utilized in an atomizing portion of an electronic cigarette (a part configured to generate vapor from a source liquid) combines the functions of heating and liquid delivery, by being both electrically conductive (resistive) and porous. Note here that reference to being electrically conductive (resistive) refers to components which have the capacity to generate heat in response to the flow of electrical current therein. Such flow could be imparted by via so-called resistive heating or induction heating. An example of a suitable material for this is an electrically conductive material such as a metal or metal alloy formed into a sheet-like form, i.e. a planar shape with a thickness many times smaller than its length or breadth. Examples in this regard may be a mesh, web, grill and the like. The mesh may be formed from metal wires or fibers which are woven together, or alternatively aggregated into a non-woven structure. For example, fibers may be aggregated by sintering, in which heat and/or pressure are applied to a collection of metal fibers to compact them into a single porous mass.

These structures can give appropriately sized voids and interstices between the metal fibers to provide a capillary force for wicking liquid. Thus, these structures can also be considered to be porous since they provide for the uptake and distribution of liquid. Moreover, due to the presence of voids and interstices between the metal fibers, it is possible for air to permeate through said structures. Also, the metal is electrically conductive and therefore suitable for resistive heating, whereby electrical current flowing through a material with electrical resistance generates heat. Structures of this type are not limited to metals, however; other conductive materials may be formed into fibers and made into mesh, grill or web structures. Examples include ceramic materials, which may or may not be doped with substances intended to tailor the physical properties of the mesh.

A planar sheet-like porous aerosol generating component of this kind can be arranged within an electronic cigarette such that it lies within the aerosol generating chamber forming part of an airflow channel. The aerosol generating component may be oriented within the chamber such that air flow though the chamber may flow in a surface direction, i.e. substantially parallel to the plane of the generally planar sheet-like aerosol generating component. An example of such a configuration can be found in WO2010/045670 and WO2010/045671, the contents of which are incorporated herein in their entirety by reference. Air can thence flow over both sides of the heating element, and gather vapor. Aerosol generation is thereby made very effective. In alternative examples, the aerosol generating component may be oriented within the chamber such that air flow though the chamber may flow in a direction which is substantially transverse to the surface direction, i.e. substantially orthogonally to the plane of the generally planar sheet-like aerosol generating component. An example of such a configuration can be found in WO2018/211252, the contents of which are incorporated herein in its entirety by reference.

The aerosol generating component may have any one of the following structures: a woven or weave structure, mesh structure, fabric structure, open-pored fiber structure, open-pored sintered structure, open-pored foam or open-pored deposition structure. Said structures are suitable in particular for providing a aerosol generating component with a high degree of porosity. A high degree of porosity may ensure that the heat produced by the aerosol generating component is predominately used for evaporating the liquid and high efficiency can be obtained. A porosity of greater than 50% may be envisaged with said structures. In one embodiment, the porosity of the aerosol generating component is 50% or greater, 60% or greater, 70% or greater. The open-pored fiber structure can consist, for example, of a non-woven fabric which can be arbitrarily compacted, and can additionally be sintered in order to improve the cohesion. The open-pored sintered structure can consist, for example, of a granular, fibrous or flocculent sintered composite produced by a film casting process. The open-pored deposition structure can be produced, for example, by a CVD process, PVD process or by flame spraying. Open-pored foams are in principle commercially available and are also obtainable in a thin, fine-pored design.

In one embodiment, the aerosol generating component has at least two layers, wherein the layers contain at least one of the following structures: a plate, foil, paper, mesh, woven structure, fabric, open-pored fiber structure, open-pored sintered structure, open-pored foam or open-pored deposition structure. For example, the aerosol generating component can be formed by an electric heating resistor consisting of a metal foil combined with a structure comprising a capillary structure. Where the aerosol generating component is considered to be formed from a single layer, such a layer may be formed from a metal wire fabric, or from a non-woven metal fiber fabric. Individual layers are advantageously but not necessarily connected to one another by a heat treatment, such as sintering or welding. For example, the aerosol generating component can be designed as a sintered composite consisting of a stainless steel foil and one or more layers of a stainless steel wire fabric (material, for example AISI 304 or AISI 316). Alternatively the aerosol generating component can be designed as a sintered composite consisting of at least two layers of a stainless steel wire fabric.. The layers may be connected to one another by spot welding or resistance welding. Individual layers may also be connected to one another mechanically. For instance, a double-layer wire fabric could be produced just by folding a single layer. Instead of stainless steel, use may also be made, by way of example, of heating conductor alloys-in particular NiCr alloys and CrFeAl alloys (“Kanthal”) which have an even higher specific electric resistance than stainless steel. The material connection between the layers is obtained by the heat treatment, as a result of which the layers maintain contact with one another-even under adverse conditions, for example during heating by the aerosol generating component and resultantly induced thermal expansions. Alternatively, the aerosol generating component may be formed from sintering a plurality of individual fibers together. This, the aerosol generating component can be comprised of sintered fibers, such as sintered metal fibers.

The aerosol generating component may comprise, for example, an electrically conductive thin layer of electrically resistive material, such as platinum, nickel, molybdenum, tungsten or tantalum, said thin layer being applied to a surface of the vaporizer by a PVD or CVD process, or any other suitable process. In this case, the aerosol generating component may comprise an electrically insulating material, for example of ceramic. Examples of suitable electrically resistive material include stainless steels, such as AISI 304 or AISI 316, and heating conductor alloys-in particular NiCr alloys and CrFeAl alloys (“Kanthal”), such as DIN material number 2,4658, 2,4867, 2,4869, 2,4872, 1,4843, 1,4860, 1,4725, 1,4765 and 1,4767.

As described above, the aerosol generating component may be formed from a sintered metal fiber material and may be in the form of a sheet. Material of this sort can be thought of a mesh or irregular grid, and is created by sintering together a randomly aligned arrangement or array of spaced apart metal fibers or strands. A single layer of fibers might be used, or several layers, for example up to five layers. As an example, the metal fibers may have a diameter of 8 to 12 µm, arranged to give a sheet of thickness 0.16 mm, and spaced to produce a material density of from 100 g/m² to 1500 g/m², such as from 150 g/m² to 1000 g/m², 200 g/m² to 500 g/m², or 200 to 250 g/m², and a porosity of 84%. The sheet thickness may also range from 0.1 mm to 0.2 mm, such as 0.1 mm to 0.15 mm. Specific thicknesses include 0.10 mm, 0.11 mm, 0.12 mm, 0.13 mm, 0.14 mm, 0.15 mm or 0.1 mm. Generally, the aerosol generating component has a uniform thickness. However, it will be appreciated from the discussion below that the thickness of the aerosol generating component may also vary. This may be due, for example, to some parts of the aerosol generating component having undergone compression. Different fiber diameters and thicknesses may be selected to vary the porosity of the aerosol generating component. For example, the aerosol generating component may have a porosity of 66% or greater, or 70% or greater, or 75% or greater, or 80% or greater or 85% or greater, or 86% or greater.

The aerosol generating component may form a generally flat structure, comprising first and second surfaces. The generally flat structure may take the form of any two dimensional shape, for example, circular, semi-circular, triangular, square, rectangular and/ or polygonal. Generally, the aerosol generating component has a uniform thickness.

A width and/or length of the aerosol generating component may be from about 1 mm to about 50 mm. For example, the width and/or length of the vaporizer may be from 1 mm, 2 mm, 3 mm, 4 mm, 5 mm, 6 mm, 7 mm, 8 mm, 9 mm or 10 mm. The width may generally be smaller than the length of the aerosol generating component.

Where the aerosol generating component is formed from an electrically resistive material, electrical current is permitted to flow through the aerosol generating component so as to generate heat (so called Joule heating). In this regard, the electrical resistance of the aerosol generating component can be selected appropriately. For example, the aerosol generating component may have an electrical resistance of 2 ohms or less, such as 1.8 ohms or less, such as 1.7 ohms or less, such as 1.6 ohms or less, such as 1.5 ohms or less, such as 1.4 ohms or less, such as 1.3 ohms or less, such as 1.2 ohms or less, such as 1.1 ohms or less, such as 1.0 ohm or less, such as 0.9 ohms or less, such as 0.8 ohms or less, such as 0.7 ohms or less, such as 0.6 ohms or less, such as 0.5 ohms or less. The parameters of the aerosol generating component, such as material, thickness, width, length, porosity etc. can be selected so as to provide the desired resistance. In this regard, a relatively lower resistance will facilitate higher power draw from the power source, which can be advantageous in producing a high rate of aerosolization. On the other hand, the resistance should not be so low so as to prejudice the integrity of the aerosol generator. For example, the resistance may not be lower than 0.5 ohms.

Planar aerosol generating components, such as heating elements, suitable for use in systems, devices and articles disclosed herein may be formed by stamping or cutting (such as laser cutting) the required shape from a larger sheet of porous material. This may include stamping out, cutting away or otherwise removing material to create openings in the aerosol generating component. These openings can influence both the ability for air to pass through the aerosol generating component and the propensity for electrical current to flow in certain areas.

The reservoir of aerosolizable material can take on any shape and in some examples forms an annular shape surrounding the aerosol generation chamber and divided therefrom by a wall. The heating element generally extends across the aerosol generation chamber and is supported in place by its edges passing through the dividing wall or resting in gaps in the wall. In this way, edge portions of the heating element can be positioned in contact with the reservoir interior and can collect liquid therefrom by capillary action. This liquid is then drawn into more central portions of the heating element. Electrical connections are provided on the heating element which enable the passage of electrical current, producing the required heating to vaporize the liquid held in the porous structure of the heating element. Vapor is delivered into the aerosol generation chamber for collection by the flow of air along the airflow channel. Alternatively, as mentioned above, the heating current may comprise eddy currents generated by electromagnetic induction, requiring an electromagnet to produce a rapidly alternating magnetic field penetrating the aerosol generating component.

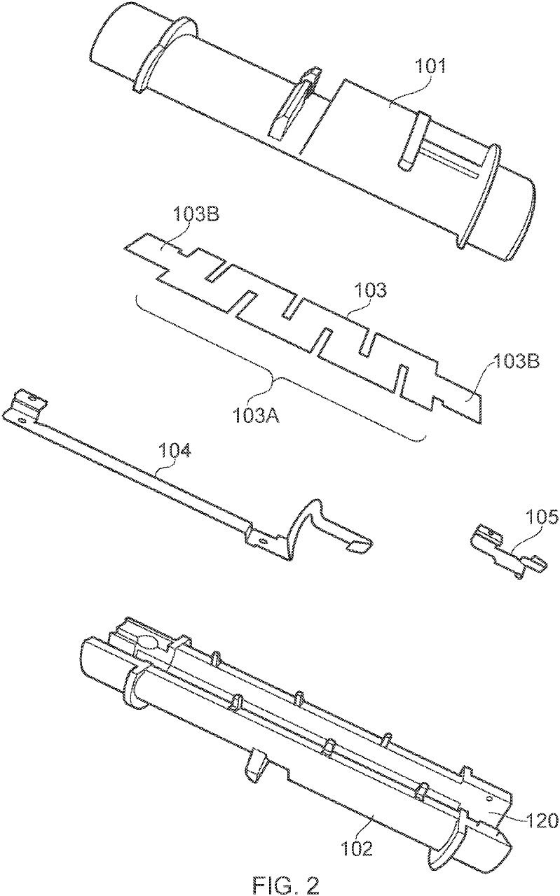

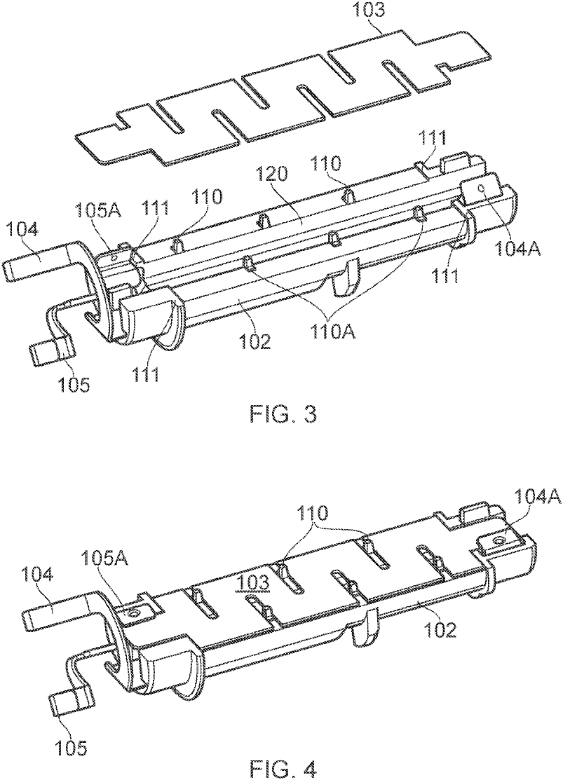

FIG. 2 shows an exploded perspective view of various components of an example atomizer of this format. FIGS. 3 to 6 show perspective views of the components represented in FIG. 2 at different stages of assembly.

The atomizer 160 comprises a first carrier component (first part) 101 and a second carrier component (second part) 102. These two components 101, 102 play a role in supporting a planar heating element 103, and in this regard may sometimes be referred to as providing a heating element cradle. Thus, the first and second components 101, 102 represented in FIG. 2 may for convenience, and having regard to the orientation represented in the figures, also be referred to as an upper cradle 101 and a lower cradle 102. The atomizer 160 further comprises the heating element 103, a first electrical contact element 104 for connecting to a first end of the heating element 103 and a second electrical contact element 105 for connecting to a second end of the heating element 103.

The upper and lower cradle components 101, 102 may be molded from a plastics material having a high glass fiber content (e.g. 50%) to provide improved rigidity and resistance to high temperatures, for example temperatures around 230° Cntigrade. The respective upper and lower cradle components are broadly speaking of a generally semi-circular cross-section (although with variations in size and shape along their lengths as discussed further below). Each cradle component is provided with a recess 120 (only visible for lower cradle component 102 in FIG. 2) running along its length on what would otherwise be their flattest faces so that when the two cradle components are brought together to sandwich the heating element 103 as discussed further below they form a cradle having a generally tubular configuration with an airflow path (defined by the respective recesses 120) running down the interior of the tube and in which the heating element 103 is disposed. The airflow path formed by the two recessed 120 comprises the aerosol generation chamber of the atomizer 160. It is noted that the cradle need not take on an elongate form, but may have width and length dimensions which are similar. Moreover, the dimensions of the respective recesses 120 may be varied as described further below.

The first and second electrical contact elements 104, 105 may be formed of a sheet metal material, for example comprising copper strips formed into an appropriate shape having regard to the shape and configuration of the other elements of the apparatus in accordance with conventional manufacturing techniques, or may comprise conventional flexible wiring. Of course, in examples where electrical energy is inductively coupled to the heating element it will be understood that such contact elements are not required.

The planar heating element 103 is formed from a sintered metal fiber material and is generally in the form of a sheet. However, it will be appreciated that other porous conducting materials may equally be used. In this particular example, the heating element 103 comprises a main portion 103A with electrical contact extensions 103B at each end for connecting to the respective electrical contact elements 104, 105. In this example, the main portion 103A of the heating element is generally rectangular with a longitudinal dimension (i.e. in a direction running between the electrical contact extensions 103B) of around 20 mm, and a width of around 8 mm.

In the example of FIG. 2, the longitudinal dimension corresponds to the direction of airflow through the vaporization chamber (note that in other examples, the longitudinal dimension need not be the longest dimension of the heating element). The thickness of the sheet comprising the heating element 103 in this example is around 0.15 mm. As can be seen in FIG. 2, the generally rectangular main portion 103A of the heating element 103 has a plurality of openings in the form of slots extending inwardly from each of the longer sides (sides parallel to the longitudinal direction). The slots extend inwardly by around 4.8 mm and have a width of around 0.6 mm. The slots extending inwardly are separated from one another by around 5.4 mm on each side of the heating element with the slots extending inwardly from the opposing sides being offset from one another by around half this spacing. In other words, the slots are alternately positioned along the longitudinal sides. A consequence of this arrangement of slots in the heating element is that current flow along the heating element is in effect forced to follow a meandering path which results in a concentration of current, and hence electrical power, around the ends of the slots. In this regard, and due to the presence of the slots, the heating element has been constructed such that some areas of the heating element (in this example the meandering path) have a greater propensity for current flow than others.

The different current / power densities at different locations on the heating element give areas of relatively high current density that become hotter than areas of relatively low current density. This provides the heating element with a range of different temperatures and increases temperature gradients, which can be desirable in the context of aerosol provision systems. This is because different components of a source liquid may aerosolize vaporize at different temperatures, so providing a heating element with a range of temperatures can help simultaneously aerosolize a range of different components in the source liquid.

A process of assembling the components represented in FIG. 2 to provide an atomizer 160 such as for use in a cartridge assembly 30 of an electronic cigarette 10 is now described with reference to FIGS. 3 to 6.

As can be seen in FIG. 3, the first and second electrical contact elements 104, 105 have been mounted to the lower cradle component 102 and the heating element 103 is represented above the lower cradle component 102 ready to be put in place. The second electrical contact element 105 is mounted at a second end of the lower cradle component 102 (the leftmost end for the orientation in FIG. 3). One end of the second electrical contact element 105 provides a second electrical contact element clamp portion 105A for receiving one of the electrical contact extensions 103B of the heating element 103 while the other end of the second electrical contact element 105 extends away from the lower cradle component 102 as shown in the figure. The first electrical contact element 104 is mounted so as to run along the length of the lower cradle component 102 adjacent a wall of the recess 120. One end of the first electrical contact element 104 extends away from the second end of the lower cradle component 102 as schematically represented in the figure. The other end of the first electrical contact element 104 provides a first electrical contact element clamp portion 105A arranged at a first end of the lower cradle component 102 (rightmost end in FIG. 3) for receiving the other of the electrical contact extensions 103B of the heating element 103.

An upper surface of the lower cradle component 102 comprises a plurality of locating pegs 110 which align with the slots in the heating element discussed above and corresponding locating holes in the upper cradle 101 (not shown in the figures). Although not essential, these locating pegs are for helping to align the upper cradle 101 with the lower cradle 102, and for helping to align the heating element 103 relative to the upper and lower cradles 102 when assembled.

FIG. 4 shows the heating element 103 mounted to the lower cradle 102 containing the first and second electrical contact elements 104, 105. The heating element 103 is mounted to the lower cradle simply by being placed on the upper surface of the lower cradle 102 with the locating pegs 110 aligned with the slots of the heating element 103. Slightly raised portions of the upper surface of the lower cradle element 102 provide locating walls 111 in the vicinity of the electrical contact extensions 103B at each end of the heating element 103 to further help align the heating element. In this example, the locating walls are separated by slightly more than the size of the heating element and the locating pegs are slightly smaller than the size of the slots so the heating element is overall free to move slightly in the horizontal plane, for example by around 0.1 mm. This is to allow for thermal expansion and contraction when the heating element is in use to help avoid buckling. The first and second electrical contact element clamping portions 104A, 105A are bent down so as to clamp around respective ones of the electrical contact extensions 103B at each end of the heating element 103, thus providing an electrical connection between the portions of the electrical contact elements 104, 105 extending away from the lower cradle component 102 and the ends of the heating element 103. In this example the electrical connections between the electrical contact elements 104, 105 and the heating element 103 rely solely on physical contact, but in other implementations other techniques may be used, for example welding or soldering.

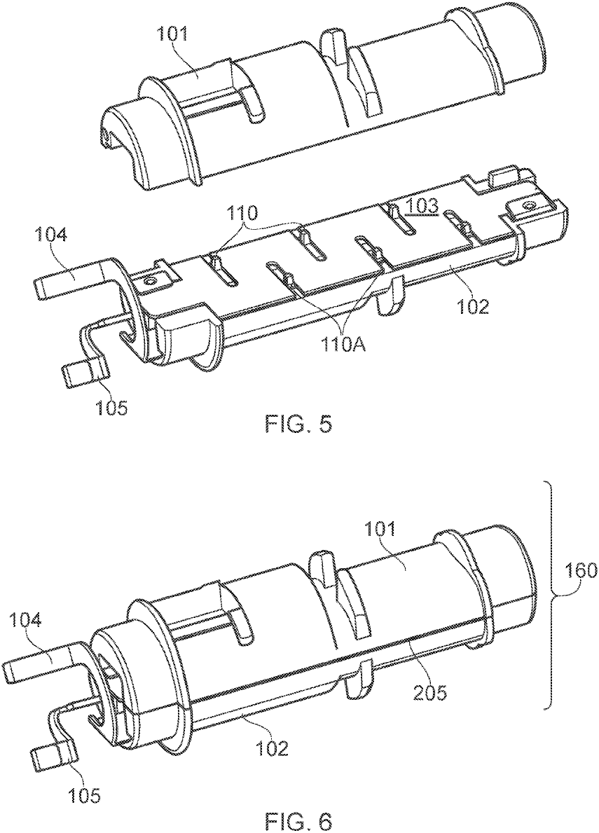

FIG. 5 shows the combined lower cradle component 102, first and second electrical contact elements 104, 105 and the heating element 103 as represented in FIG. 4, but with the other cradle component 101 shown ready to be mounted to the lower cradle component.

FIG. 6 schematically shows the upper cradle component 101 mounted to the lower cradle component 102 (and other elements represented in FIG. 4) to provide an assembled atomizer 160. The upper cradle component 101 is mounted to the lower cradle component 102 by simply placing them together with the locating pegs 110 of the lower cradle component aligned with corresponding locating holes (not shown) in the upper cradle component 101. As can be seen in FIGS. 4 and 5, the locating pegs 110 are each provided with a shoulder 110A. The shoulders 110A have a height above the upper surface of the lower cradle component 102 that matches the height of the locating walls 111 but is slightly larger than the thickness of the heating element 103. The shoulders 110A are sized and arranged so as to fall within the slots of the heating element. However, the corresponding locating holes in the upper cradle are sized only to receive the locating pegs, and not their shoulders. Thus, when the upper cradle component 101 is mounted to the lower cradle component 102 they are separated by a gap 205 corresponding to the height of the shoulders 110A and the locating walls 111. The gap is greater than the thickness of the heating element, so the heating element is loosely sandwiched between the upper and lower cradle components, rather than being fixedly clamped in place. As noted above, this loose mounting of the heating element is to allow for thermal expansion and contraction of the heating element during use.

Thus, the assembled atomizer 160 is generally tubular with a central passageway forming an aerosol generation chamber defined by the respective recesses 120 in the upper and lower carrier components, providing an airflow path through the atomizer that will connect to an air inlet and an air outlet in a complete electronic cigarette. In use, the particular atomizer 160 of FIG. 6 is annularly surrounded by the reservoir of source liquid. The gap 205 is in fluid communication with the reservoir and hence provides a capillary channel (one each side) which extends along both sides of the heating element 103 and through which aerosolizable material may be drawn from the reservoir to the heating element where it enters the pores of the heating element for vaporization to generate a vapor in the aerosol generation chamber 120 during use. The passing air collects the vapor to generate an aerosol to be drawn out of the vaporization chamber and along a further part of the airflow path through the electronic cigarette 10 to exit through the air outlet as a user inhales on the electronic cigarette 10.

When installed in an electronic cigarette, an atomizer may be arranged such that the longitudinal dimension of the heating element, corresponding to the direction of airflow through the atomizer from the upstream to downstream ends, is aligned parallel to the longitudinal axis of the electronic cigarette for an end-to-end device such as the FIG. 1 example, or at least the longitudinal axis of the cartridge component in a side-by-side device having a power component arranged to the side of a cartridge component. This is not compulsory, however, and in the current description, the term “longitudinal” is intended to refer to the dimensions and orientation of the atomizer, in particular the dimension of the heating element along the airflow path from an atomizer inlet at the upstream end of the atomizer, and through the vaporization chamber to the atomizer outlet at the downstream end of the atomizer.

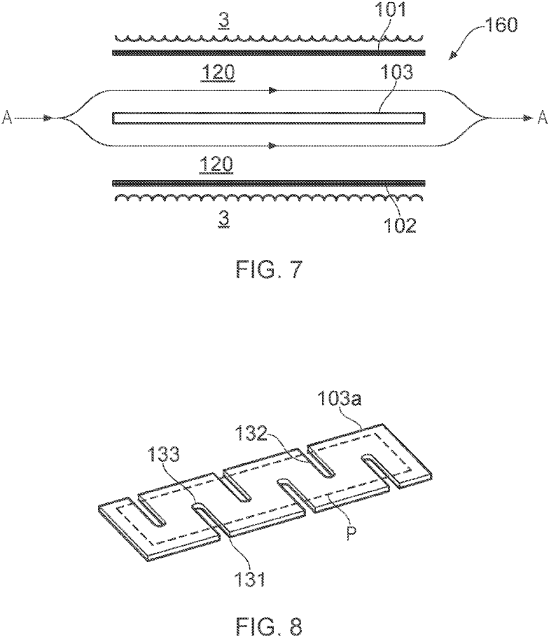

FIG. 7 shows a highly simplified longitudinal cross-sectional side view of the example atomizer 160 in use, where the section is orthogonal to the plane of the heating element 103. The upper and lower cradle components 101 and 102 (or similar housing to form the aerosol generation chamber and support the heater) form outer walls which divide the interior of the atomizer 160 from the surrounding reservoir 3. The interior forms the aerosol generation chamber 120. The heating element 103, which is shown edge-on, extends longitudinally through the vaporization chamber 120, and generates vapor into the aerosol generation chamber as discussed. An upstream end (shown left) of the aerosol generation chamber 120 connects with an upstream part of the airflow channel through the electronic cigarette, leading from one or more air inlets (not shown in FIGS. 6 and 7). A downstream end (shown right) of the vaporization chamber 120 connects with a downstream part of the airflow channel, leading to the mouthpiece air outlet. Consequently, when a user inhales through the air outlet, air drawn in through the inlet(s) enters the aerosol generation chamber 120 and follows a longitudinal path, able to flow over both surfaces of the planar heating element 103 before recombining at the far end to travel on to the air outlet. This is shown by the arrows A in the figure. Accordingly, the path length through the aerosol generation chamber 120 and over the heating element surfaces is relatively long, comprising effectively the full length of the heating element 103. The flowing air is hence able to collect a large amount of vapor, which condenses to form aerosol droplets. The airflow path through the atomizer 160 can influence the formation of aerosol in the aerosolization chamber and it has been found that certain configurations of the aerosol generating component (heater) with respect to the aerosol generating chamber can lead to variations in the size of aerosol particles formed. Such variations in particle size may lead to aerosols which are more acceptable to consumers.

There will now be described an aspect of the present disclosure relating to the supply of aerosolizable material to the aerosol generating component. In particular, this aspect relates to an article for use with an electrically operated non-combustible aerosol delivery system, the article comprising a generally planar aerosol generating component suspended within an aerosol generating chamber, wherein the periphery of the aerosol generating component is coupled to one or more feed apertures such that liquid aerosolizable material may be fed directly to the majority of the periphery.

In this regard, liquid being “fed directly” means that liquid reaching the periphery does so from a location outwardly, e.g. radially outwardly, of the periphery, rather than reaching the periphery as a result of internal movement from another location within the aerosol generating component.

In this regard, it is also noted that reference is generally made to liquid aerosolizable material being fed to the aerosol generating component in the context of this aspect. This does not preclude the aerosolizable material being in another state, e.g. a gel, in another part of the system and being converted to liquid for delivery to the aerosol generating component.

In some embodiments, the aerosolizable material may be fed directly to substantially all of the periphery of the aerosol generating component. For example, the aerosolizable material may be fed directly to 80% or more of the periphery of the aerosol generating component. For example, the aerosolizable material may be fed directly to 90% or more of the periphery of the aerosol generating component. For example, the aerosolizable material may be fed directly to 95% or more of the periphery of the aerosol generating component. In some embodiments, the aerosolizable material may be fed directly to the entirety of the periphery of the aerosol generating component.

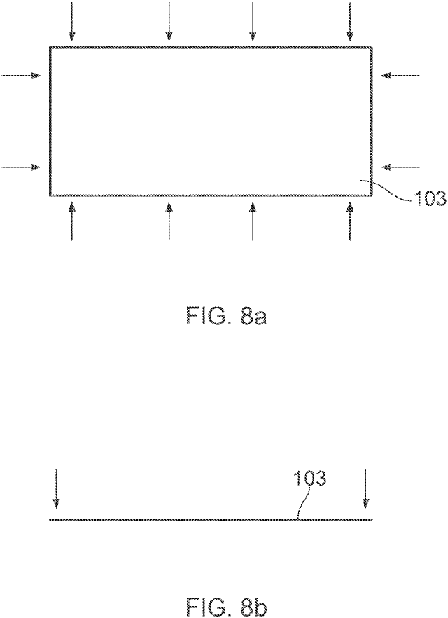

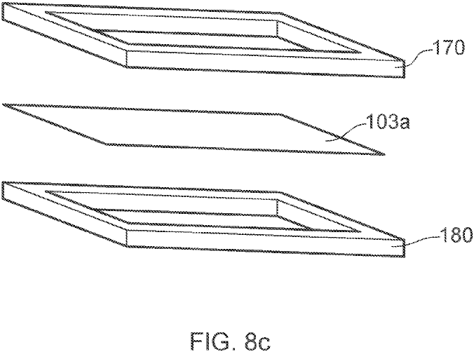

FIG. 8 shows a heating element 103a. Heating element 103a may be to be fed with aerosolizable material around the majority of its periphery. In this regard, the periphery of the heating element 103a is considered to be, for the heating element 103a, the broadly rectangular outer profile shown by dotted line P excluding the perimeter sections which form the slots in the heating element. Indeed, although heating element 103a includes slots, they are not essential in the context of the present aspect. As a result of being arranged to receive aerosolizable material around the majority of its periphery, it is possible to increase the feeding of aerosolizable material to the heating element 103a relative to other heating elements which are only fed via one or two sides (such as the heating element 103 referred to in FIGS. 2 to 7).

The ability to feed aerosolizable material around the periphery of the heating element 103a has implications for the design of the cradle sections, the coupling of electrical energy to the heating element, and also the aerosol generating chamber of the article. For example, electrical contact with the heating element may be effected through means other than the electrical contact extensions 103B, such as via electrical contacts embedded in the cradle sections as appropriate. Alternatively, where energy is inductively coupled to the heating element it will be understood that no electrical contacts are in fact required, and this is another advantage of configuring the aerosol generating component such that it is configured to be fed with aerosolizable material around its entire periphery.

Delivery of the aerosolizable material directly to the periphery of the aerosol generating component may be achieved in a number of ways. In some embodiments, the aerosolizable material is fed to the periphery of the aerosol generating component 103 in a plane which is parallel to the plane of the aerosol generating component. Such a feed arrangement is shown in FIG. 8a. In some embodiments, the aerosolizable material is fed to the periphery of the aerosol generating component 103 substantially orthogonally to the plane of the aerosol generating component. Such a feed arrangement is shown in FIG. 8b.

In some examples, the at least one feed aperture is a capillary gap. The capillary gap may be formed in a wall of the aerosol generating chamber. The capillary gap may extend around the aerosol generating chamber in a plane parallel to the plane of the aerosol generating component. In some examples, the capillary gap extends around at least 90% of the perimeter of the aerosol generating component. The capillary gap may extend intermittently around the aerosol generating chamber. Alternatively, the capillary gap may extend continuously around the aerosol generating chamber.

FIG. 8c shows an embodiment whereby the aerosol generating component is fed with aerosolizable material via a capillary gap. In particular, aerosol generating component 103a is shown has having a generally rectangular profile. Above and below aerosol generating component 103a are capillary forming elements 170 and 180. Each capillary element has a profile which generally corresponds to that of the aerosol generating component 103a. Thus, in this example the profile is generally rectangular, but it will be understood that when the profile of the aerosol generating component varies then the profile of the capillary forming elements 170, 180 can vary accordingly. In a similar fashion to that described above with respect to cradles 101 and 102, in use capillary forming elements 170 and 180 are arranged just above and below the aerosol generating component 103a such that the perimeter of the aerosol generating component 103a is located in a capillary gap formed by the capillary elements. Due to the profile of the capillary elements generally corresponding to that of the aerosol generating component 103a, the capillary gap can extend around the entire periphery of the aerosol generating component. This can allow for liquid to be fed to the entire periphery of the aerosol generating component 103a.

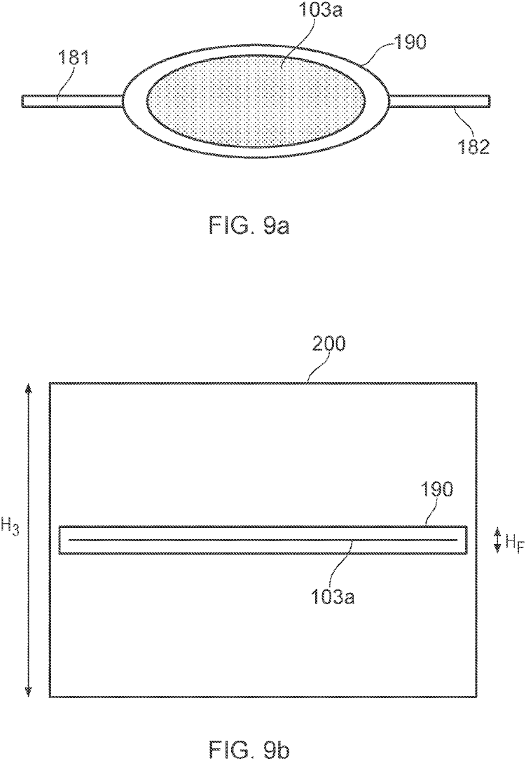

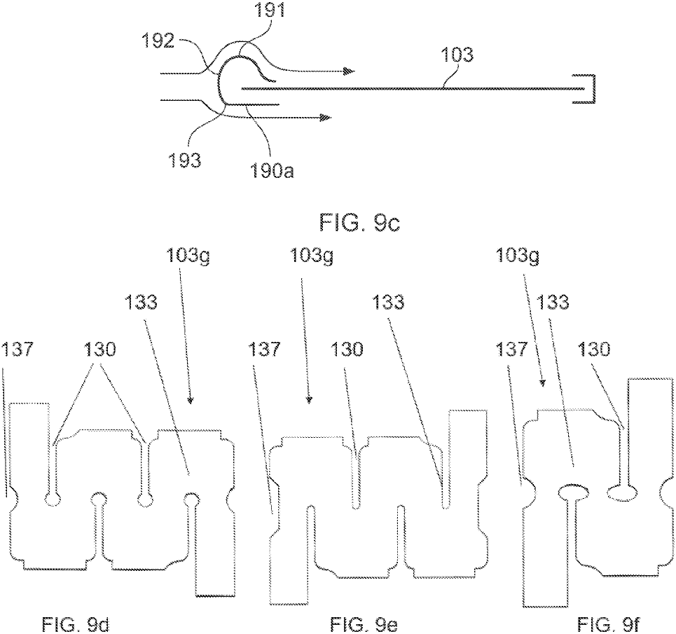

Capillary forming elements 170 and 180 can form part of the wall which defines the aerosol generating chamber, or can alternatively form part of a capillary frame which is located within the aerosol generating chamber. The capillary frame might take any shape conforming to the perimeter of the aerosol generating component. For example, where the aerosol generating component is circular, the frame may take the form of a torus extending around the perimeter of the aerosol generating component. For example, as shown in FIG. 9a, the aerosol generating component 103a is located within a capillary frame 190. The aerosol generating component 103a extends into a capillary gap (not shown) formed in the frame. The frame is then fed with aerosolizable material via one or more capillary conduits 181, 182. The capillary conduits 181,182 are in fluid communication with the system reservoir and thus provide a route for liquid aerosolizable material to travel from the reservoir to the capillary frame and thus to the aerosol generating component. By locating the aerosol generating component in such a capillary frame it is possible to ensure that liquid is fed directly to the entire periphery of the aerosol generating component, whilst at the same time allowing for airflow to be channeled across the surface of the aerosol generating component 103a. Such a frame 190 (which can be a single piece or formed from two or more capillary frame elements) can be located within an aerosol generating chamber 200. In order to ensure that the frame does not significantly disrupt the airflow over the aerosol generating component, it may be that the total frame thickness H_{F} is less than 20% of the total height H₃ of the aerosol generating chamber 200 as is illustrated in FIG. 9b. In some embodiments, the frame height is minimized so as to cause a minimal amount of disruption to the airflow flowing across the aerosol generating component.

In some examples where the air flow is across the surface of the aerosol generating component such that it has to by-pass the capillary frame, the frame may have a profile which influences the velocity of the airflow across it. For example, as shown in FIG. 9c, frame 190a is provided with a leading face 192. Face 192 projects towards air entering the aerosol generating chamber and thus the geometry of face 192 can influence the resultant downstream flow of air. In FIG. 9c, face 192 is shown as having a profile similar to that of the leading edge of an aircraft wing, e.g. it may be configured as an aerofoil. As incoming air (shown by the arrows in FIG. 9c) flows over the face 192, the different in profile between section 191 and 193 of the face 192 results in air travelling at different speeds. By changing the profile of the face 192 it is possible to influence the speed of the airflow over the aerosol generating component. Accordingly, in one broad aspect, there is provided an article comprising an aerosol generating component suspended within an aerosol generating chamber, wherein airflow travelling through the chamber and over the surface of the aerosol generating component has been influenced by an aerofoil shaped component. It will be appreciated that in FIG. 9c the aerofoil shaped component is the leading edge of the capillary frame but it will be appreciated that other aerofoil shaped components could be located within the chamber/at the air inlet so as to influence the airflow. It is also possible that the trailing edge of the capillary frame may be provided with a profile which modifies the velocity of the airflow across it.

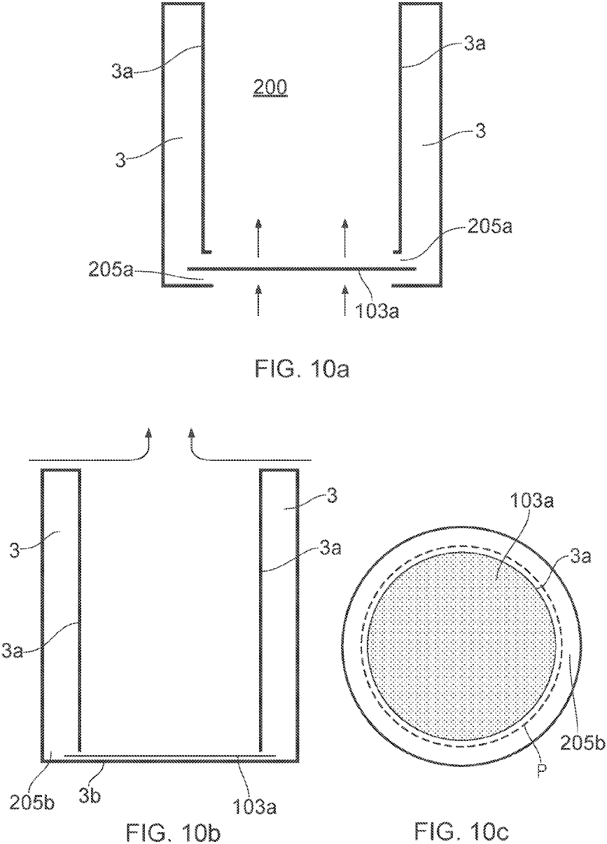

In other examples, the at least one feed aperture is part of the wall of the aerosol generating chamber. FIG. 10a shows a cross-section through an annular reservoir 3 configured such that the central aperture through the reservoir forms the aerosol generating chamber 200. As shown in FIG. 10a, the aerosol generating component 103a can be located within a feed aperture forming a capillary gap 205a which is in fluid communication with the reservoir 3. The internal walls of the reservoir 3a also serve to form the aerosol generating chamber 200 which forms part of the airflow channel through the device. It will be understood that in this embodiment airflow is configured to pass through the aerosol generating component rather than across its surface as shown by the direction of the arrows.

It is also envisaged that the at least one feed aperture could be located outside of the plane of the aerosol generating component. For example, and as illustrated schematically in FIGS. 10b and 10c, the aerosol generating component 103a is located at the base of the reservoir 3 such that a feed aperture 205b is located in proximity to the periphery P of the aerosol generating component 103a. This arrangement allows for direct gravity feeding to the periphery of the aerosol generating component without the need to arrange the feed aperture in the form of a capillary gap. This can be advantageous as lesser manufacturing tolerances are required when forming the gravity fed aperture compared to the capillary gap. In FIG. 10b, the reservoir has been depicted having a base 3b. This can be useful so as to minimize leakage from the reservoir. Airflow in this embodiment can be arranged by providing the base 3b with one or more airflow apertures (not shown), in which case they could be equated to air inlets into the aerosol generating chamber. Alternatively, air flow can be directed so that air passes past the ends of the reservoir 3 so as to entrain aerosol as shown by the arrows in FIG. 10b.

It is also envisaged that the feed of aerosolizable material can be correlated to certain areas of the aerosol generating component. Thus, in another aspect of the present disclosure, the aerosol generating component is fluidly coupled to at least one component for transferring aerosolizable material to the aerosol generating component such that aerosolizable material is preferentially delivered to portions of the aerosol generating component configured to vaporize the aerosolizable material at a higher rate than other areas of the aerosol generating component. For example, aerosolizable material is preferentially fed to those areas of the aerosol generating component which are configured to dissipate higher energy during use, e.g. in the form of higher temperature. Such higher energy dissipation may result from more power being provided to a particular portion of the aerosol generating component. Such areas of greater power will have a propensity to convert the aerosolizable material to a vapor more rapidly than those areas with a lower power. Thus and where for example aerosolization occurs via resistive heating, by matching the feeding of aerosolizable material to those areas with the greatest rate of vaporization, a more efficient system can be provided with less risk of overheating the aerosolizable material.

In some examples, the at least one component for transferring aerosolizable material is selected from a wick, a pump, or a capillary gap. In some examples, the portions of the aerosol generating configured to vaporize the aerosolizable material at a higher rate than other areas of the aerosol generating component, are areas which are configured to be heated to a higher temperature during use. An example of an aerosol generating component configured such that some areas/portions are configured to dissipate more energy during use compared to other areas/portions is an aerosol generating component having portions with a relatively greater propensity for flow of electrical current. When an electrical current is passed through such an aerosol generating component, current will preferentially flow through those portions having a high propensity for electrical current flow. This will lead to greater resistive heating in those areas compared to others and thus greater power dissipation due to P = I²R.

In some examples, the portions of greater and lesser propensity for current flow are generated by creating areas of extremely high resistance in the aerosol generating component. For example, the aerosol generating component can be provided with apertures or slots (as described above) which effectively serve to prevent current flow. If, for example, such apertures or slots extend from the perimeter of the aerosol generating component, electrical current will preferentially flow through more central regions of the aerosol generating component and those areas will be subject to greater resistive heating.

In some examples where the aerosol generating component has portions of greater and lesser rates of vaporization, this may result from the portions having different densities.

In some examples, the portions having greater and lesser propensity for the flow of electrical current are formed from different materials.

The particular shape of the portions having greater or lesser propensity for current flow (or indeed rate of vaporization in the broadest aspect) is not particularly limited and can be chosen based on other factors such as the configuration of the airflow through the aerosol generating chamber.

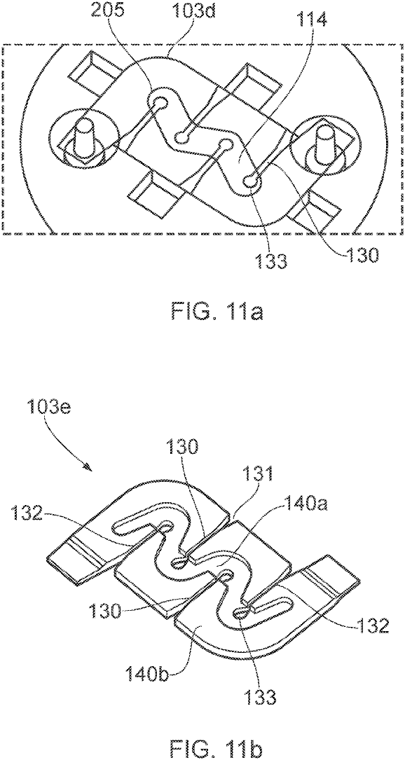

FIG. 11a shows an example of an aerosol generating component 103d having portions with a greater propensity for electrical current flow. In particular, FIG. 11a shows an aerosol generating component formed from a material such as stainless steel. The aerosol generating component 103d has a capillary structure by virtue of it being formed from a plurality of stainless steel fibers which have been sintered together such that the interstices between the fibers form a capillary structure. The capillary structure is not, however, visible in FIG. 11a. The aerosol generating component 103d comprises a plurality of slots 130. These slots are as described elsewhere in the present disclosure. It is noted that the presence of the slots is not essential and they may be omitted, but in this embodiment they serve to create portions having a greater propensity for electrical current flow. The aerosol generating component 103d also includes an area having a greater propensity for electrical current flow 114 and an area having a lesser propensity for electrical current flow 115. In the embodiment of FIG. 11a, the areas having a greater propensity for electrical current flow 114 take a serpentine shape and result from the presence of slots 130 which serve to influence the preferred flow of electrical current through the aerosol generating component 103d. In particular, the flow of electrical current will be greatest in proximity to the rounded apexes 133 of each slot 130a. The specific configuration of the slots may vary as explained more generally in the present disclosure.

As has been explained above, in some examples it can be desirable to preferentially deliver aerosolizable material to said portions having a greater propensity for current flow. This can be achieved in a number of ways. For example, a component for transferring aerosolizable material can be configured to preferentially deliver aerosolizable material to the desired areas. In some examples, the component for transferring aerosolizable material comprises a wick (or a plurality of wicks) and the (or each) wick contacts the portions having a greater propensity for current flow. In this way, aerosolizable material can be delivered directly to those portions having a greater propensity for current flow rather than having to flow through the other areas of the aerosol generating component, as is the case for the embodiment described in FIGS. 2 to 6.

Alternatively, if a pump or other mechanical means is used as the component for transferring aerosolizable material, the pump can be configured so as to deliver liquid at a faster rate to portions having a greater propensity for current flow (or indeed energy dissipation) than to other sections.

Alternatively, if a capillary gap is used as the component for transferring aerosolizable material, the capillary gap can be configured so as to have its edge located in proximity to the portions having a greater propensity for current flow. Thus, contrary to the embodiments of FIGS. 2 to 10 where aerosolizable material must flow through the outer sections of the aerosol generating component 103 in order to reach the central areas having a greater temperature, in the present embodiment it is possible for aerosolizable material to be delivered directly in proximity to portions having a greater propensity for current flow. Such an arrangement is shown in FIG. 11a. In FIG. 11a there is depicted in outline the edge of a continuous capillary gap 205. The capillary gap 205 takes a serpentine or zig-zag shape with the ends and points of inflection being located in proximity to the apexes 133 of each slot. By configuring the capillary gap 205 to track the portions having a greater propensity for current flow, it is therefore possible to allow aerosolizable material to by-pass the portions of the aerosol generating component having a lesser propensity for current flow.

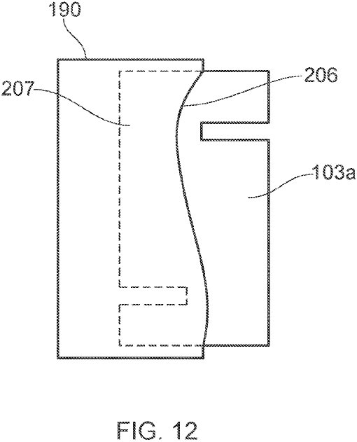

Referring now to FIG. 12, there is shown a schematic plan view of aerosol generating component 103a extending into a capillary gap 205 of a capillary frame 190. The capillary gap 205 has a mouth section 206 and a body section 207. As can be seen, the aerosol generating component 103a extends past the mouth section 206 and into the body section 207. The mouth section is formed by an edge which is non-linear. It will be appreciated that the edge of the mouth section may take other forms so as to correspond to the portions having a greater propensity for current flow. In some examples the edge may be circular (as depicted in FIG. 10c), elliptical, sinusoidal or polygonal. The periphery of the aerosol generating component 103a extends into the body 207 of the capillary gap such that the mouth section 206 of the capillary gap 205 is in proximity to the boundary between the portions having greater and lesser propensity for the flow of electrical current.

It will be understood that this aspect of the present disclosure is not limited to aerosol generating components which are heated by electrical resistance heating, but also extends to other aerosol generating components having portions with respective greater and lesser rates of vaporization.

In this regard, the portions having a greater rate of vaporization may be disposed, relative to the longitudinal axis of the aerosol generating component, inwardly of the portions having a lesser rate of vaporization. The portions having a lesser rate of vaporization may be disposed at the periphery of the aerosol generating component.

Alternatively, the portions having a greater rate of vaporization may be disposed, relative to the longitudinal axis of the aerosol generating component, outwardly of the portions having a lesser rate of vaporization. The portions having a greater rate of vaporization may be disposed at the periphery of the aerosol generating component.

The boundary between the portions having greater and lesser rates of vaporization (propensity for the flow of electrical current) may be linear or nonlinear. In some examples, the boundary is circular, elliptical, sinusoidal or polygonal.

It is further envisaged that the air inlet into the aerosol generating chamber is arranged so as to align with specific sections of the aerosol generating component. Thus, in another aspect of the present disclosure, there is provided an article for use with an electrically operated non-combustible aerosol delivery system, the article comprising a generally planar aerosol generating component suspended within an aerosol generating chamber, the aerosol generating component having a portion configured to vaporize aerosolizable material at a higher rate than other portions of the aerosol generating component, wherein said chamber has an air inlet and one or more air outlets defining a flow path therebetween, the flow path being arranged to track said portion of the aerosol generating component configured to vaporize aerosolizable material at a higher rate than other portions of the aerosol generating component.

For example, the air inlet and outlet are preferably arranged with respect to the aerosol generating component such that the flow path between the inlet and outlet is preferentially distributed over those portions of the aerosol generating component which are configured to vaporize aerosolizable material at a greater rate during use. For example, such portions may be configured to dissipate greater power during use and thus have the potential to reach a higher temperature during use (and thus which are configured to vaporize aerosolizable material at a higher rate). Such higher temperature areas will have a propensity to convert the aerosolizable material to a vapor more rapidly than those areas with a lower temperature. By preferentially arranging the flow path with those areas of the aerosol generating component with the greatest ate of vaporization, a more efficient system can be provided. In this regard, the flow path may be understood as the direct path between inlet and outlet in the sense of being the shortest linear path between then inlet and outlet, and thus may be referred to as the “direct flow path”. Reference to “arranged to track” means that the majority of the direct flow path is within the area to be tracked. For example, where the aerosol generating component and the inlet and outlet are positioned at respective longitudinal ends of the component so that the direct flow path between the inlet and outlet will extends parallel to the aerosol generating component, the direct flow path is arranged to travel directly above (or beneath) those portions of the aerosol generating component which are configured to vaporize aerosolizable material at a greater rate during use In some examples, more than 60% of the direct flow path is within the area to be tracked. In some examples, more than 65% of the direct flow path is within the area to be tracked. In some examples, more than 70% of the direct flow path is within the area to be tracked. In some examples, more than 80% of the direct flow path is within the area to be tracked. In some examples, more than 85% of the direct flow path is within the area to be tracked. In some examples, more than 90% of the direct flow path is within the area to be tracked.

Any of the above examples of aerosol generating components configured such that some areas/portions are configured to vaporize aerosolizable material faster than other areas, e.g. by reaching a higher temperature during use compared to other areas/portions, may be employed in the context of the present aspect. For example, an aerosol generating component having portions with a greater propensity for flow of electrical current may be used. As explained above, when an electrical current is passed through such an aerosol generating component, current will preferentially flow through those areas having a higher propensity for electrical current flow. This will lead to greater resistive heating in those areas compared to others.

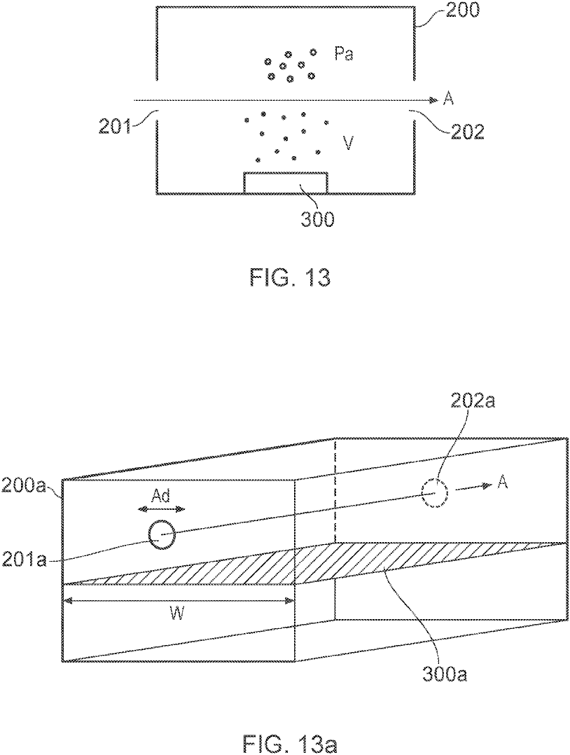

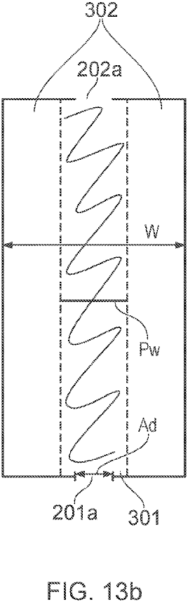

Referring in this regard to FIG. 13, an aerosol generating chamber 200 of an aerosol delivery system is shown. Generally, the aerosol delivery system comprises an aerosol generating component 300 located within, or in proximity to, the aerosol generation chamber 200. The aerosol generation chamber 200 generally comprises an inlet 201 and an outlet 202, which together facilitate airflow “A” through the aerosol generation chamber. During use of the system, aerosolizable material (not shown), is energized so as to form a vapor “V”. The produced vapor undergoes condensation in the aerosol generation chamber such that particles “Pa” are formed and entrained in the airflow through the chamber. Such particles entrained in the airflow form the aerosol of the aerosol delivery system. It will of course be appreciated that the aerosol is also composed of non-condensed vapor “V” and for particular systems there will be a particular partitioning between the particles and the vapor making up the aerosol. Thus, it is generally understood that for a condensation aerosol, there will be a “particulate phase” and a “gas or vapor phase”.

Referring now to FIG. 13a, an aerosol generating chamber 200a is shown. Similarly to aerosol generating chamber 200, aerosol generating chamber 200a comprises an air inlet 201a and an air outlet 202a. Aerosol generating component 300a is shown suspended within aerosol generating chamber 200a. In this regard, the term “suspended” generally refers to the aerosol generating component forming a bridge from one side of the aerosol generating chamber to another. As will be apparent from FIG. 13a, inlet 201a and outlet 202a are both located on the same side of the aerosol generating component. Thus, air travelling between the inlet 201a and outlet 202a does so in an orientation that is generally aligned with the longitudinal extent of the aerosol generating component 300a. Indeed, the airflow travels generally along a surface of the aerosol generating component 300a. Such an airflow configuration may be referred to as “parallel” airflow, since the airflow is generally parallel to the surface of the aerosol generating component. A similar arrangement is shown with respect to FIG. 7 above.

Air inlet 201a may take a range of shapes, as described below. However, in some examples the largest lateral dimension A_{d} of the air inlet 201a is less than the width W of the aerosol generating component 300a. Moreover, the air inlet 201a is located in the aerosol generating chamber 200d such that it is generally positioned in alignment with the portion of the aerosol generating component configured to vaporize aerosolizable material at a higher rate than other portions of the aerosol generating component. In some examples, said portion having a higher rate of vaporization is located towards the center of the aerosol generating component. In some examples, said portion having a higher rate of vaporization 301 spans a lateral extent of the aerosol generating component as shown in FIG. 13b. The portion 301 has a width P_{w} which is less than the width W of the aerosol generating component 300a. The air inlet 201a is aligned with the portion 301 such that the perimeter of air inlet 201a is within the boundaries of the portion 301. In general, P_{w} is greater than A_{d}, since this ensures that the air inlet 201a is within the portion 301.

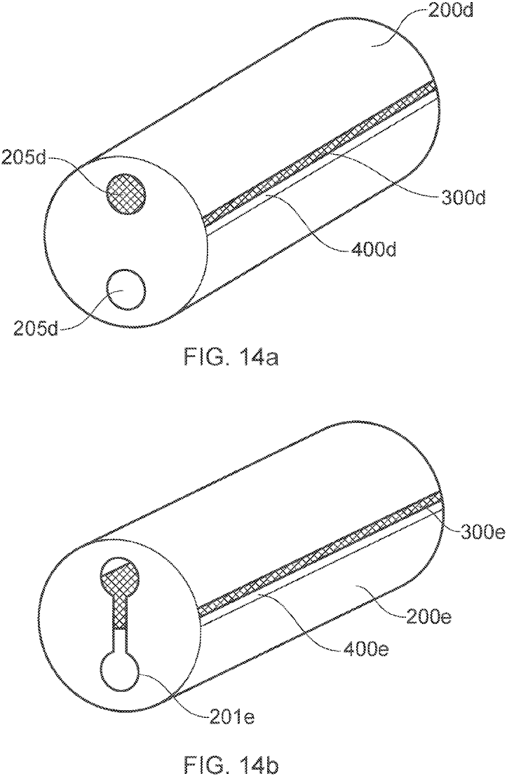

Turning to FIG. 14a, air inlet 201a in aerosol generating chamber 200d is formed by two discrete apertures 205d and 206d respectively. Aerosol generating component 300d is visible in FIG. 2a via aperture 205d. In this example, aerosol generating component 300d is located such that the surface of aerosol generating component 300d is horizontal (at 90° to a common axis through each of apertures 205d and 206d), and is equidistant from apertures 205d and 206d. Due to the displacement of apertures 205d and 206d away from the surface of the aerosol generating component, air entering chamber 200d generally does so at a distance from the surface of the aerosol generating component. Aerosol generating component 300d projects into capillary gap 400d so as to enable feeding of aerosolizable liquid from the store of aerosolizable material (as described generally above). The reservoir of aerosolizable material may surround the aerosol generating chamber, such that the walls of the aerosol generating chamber form an inner wall of the reservoir and an outer wall of the article forms an outer wall of reservoir. By ensuring air enters the chamber at a distance from the surface of the aerosol generating component, the particle size of the resulting aerosol can be controlled so as to be relatively larger than when air enters at a point which is closer to the surface of the aerosol generating component. Moreover, due to the location of the apertures 205d and 206d, the air inlets are aligned with the portion of the aerosol generating component 300d having a greater rate of vaporization.

It has also been surprisingly found that by configuring the position of the air inlet relative to the aerosol generating component, it is possible to influence the size of the particles formed in the aerosol generation chamber. Controlling the size of the particles is considered to be important in connection with aerosol delivery systems used as simulated cigarettes, such as e-cigarettes or related devices as described herein. This is because the user expects a certain sensorial experience to be associated with the use of such a system and this experience will be influenced by the size of the particles present in the aerosol. In this regard, relatively smaller particles may not be deposited greatly in the buccal cavity of the user and instead are deposited predominately further along the respiratory system. By contrast, relatively larger particles may be more likely to be deposited in the buccal cavity of the user and with relatively less deposition occurring further along the respiratory system.

Further, it has been surprisingly found that by configuring the position of the air inlet relative to the aerosol generating component, it is possible to influence the total amount of aerosol delivered by the system. Controlling the amount of aerosol delivered to the user is considered to be important in connection with aerosol delivery systems used as simulated cigarettes, such as e-cigarettes or related devices as described herein. This is because the user is able to perceive the amount of aerosol delivered per inhalation and associate a particular sensorial experience with that amount. For example, inhalations that are considered to have a relatively high amount of aerosol may be perceived by the user to provide a more fulsome mouthfeel. Additionally, for a given system and aerosolizable material, increasing the amount of aerosol per inhalation will result in an increase in the amount of active compounds delivered per inhalation. Moreover, increasing the total amount of aerosol delivered to the user per inhalation gives an indication of the efficiency of the system. Indeed, it has been surprisingly found that the position of the air inlet relative to the aerosol generating component can influence the amount of aerosol that is delivered from the system. For example, it has been found that it is possible to increase the amount of aerosol delivered by the system even though other parameters which might affect aerosol generation (such as type of aerosolizable material, power used to generate the aerosol) remain unchanged.

FIG. 14b shows a further air inlet configuration in accordance with the present disclosure. In particular, air inlet 201e is formed in aerosol generating chamber 200e. Air inlet 201e forms a single aperture which is shaped so as to bias the air entering the chamber to be distanced from the surface of the aerosol generating component. For example, air inlet takes the form of a “dumb-bell”, “dog-bone”, or “hour-glass” shape such that aperture areas of generally larger cross-sectional area are joined by an area of relatively smaller cross-sectional area. Aerosol generating component 300e projects into capillary gap 400e so as to enable feeding of aerosolizable liquid from the store of aerosolizable material (as described above). As will be appreciated, air entering the chamber will be delivered preferentially to areas which are at a greater distance along the normal from the surface of the aerosol generating component. It has been surprisingly found that by biasing air so as to be further from the surface of the aerosol generating component, the particle size can be increased.

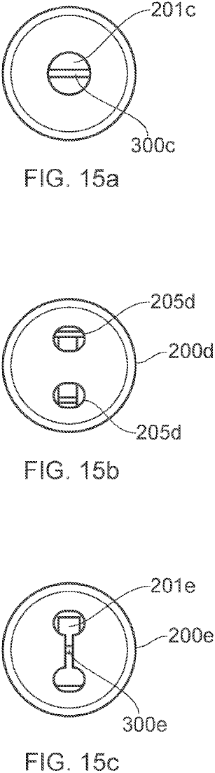

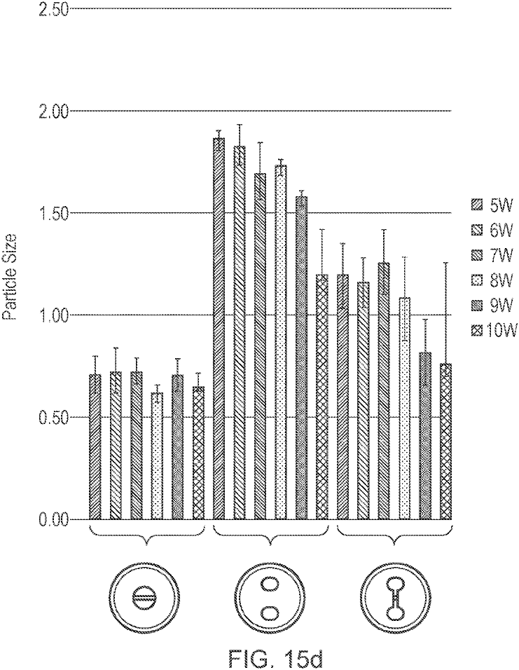

Referring now to FIG. 15, end on views of various air inlet configurations are shown. Each of FIGS. 15a, 15b and 15c show end on views of an external face of an aerosol generating chamber including an air inlet. It will be understood that in these figures the external face of the chamber has a circular cross section. However, the specific shape of the aerosol generating chamber is not limited in the context of the present disclosure. Rather, what is important is the placement of the aerosol generating component within the chamber relative to the distribution of the one or more air inlets. In particular, air inlet 201c is formed by a circular aperture and is located such that equal proportions of the aperture are distributed above and below the generally planar aerosol generating component 300c. Such a configuration leads to the majority of air entering the chamber through inlet 201c being distanced relatively nearer to the surface of the aerosol generating component. However, the air inlet 201c is still configured so as to be aligned with the portion of the aerosol generating component 300d having a greater rate of vaporization.

FIG. 15b shows an air inlet configuration in accordance with the aerosol generating chamber shown in FIG. 14a. In particular, air inlet 201d in aerosol generating chamber 200d is formed by two discrete apertures 205d and 206d respectively. Although the aerosol generating component 300d is not visible in FIG. 15b, it is located such that the surface of the aerosol generating component is horizontal and equidistant from apertures 205d and 206d. Due to the displacement of apertures 205d and 206d away from the surface of the aerosol generating component, air entering chamber 200d generally does so at a distance from the surface of the aerosol generating component. By ensuring air enters the chamber at a distance from the surface of the aerosol generating component, the particle size of the resulting aerosol can be controlled so as to be relatively larger than when air enters at a point which is closer to the surface of the aerosol generating component.

FIG. 15c shows an air inlet configuration in accordance with the aerosol generating chamber shown in FIG. 14b. In particular, air inlet 201 is formed from a single aperture which is shaped so as to bias the air entering the chamber to be distanced from surface of the aerosol generating component. For example, air inlet can take the form of a “dumbbell” or “dog-bone” shape such that areas of generally larger cross-sectional area are joined by an areas of relatively smaller cross-sectional area. As will be appreciated, air entering the chamber will be delivered preferentially to areas which are at a greater distance along the normal from the surface of the aerosol generating component. It has been surprisingly found that by biasing air so as to be further from the surface of the aerosol generating component, the particle size can be increased. In this regard, tests were conducted to assess the impact of varying the relative position of the air inlet with respect to the aerosol generating component. In particular, the particle size (D50) of an aerosol produced from an electrically heated aerosol generating component as described generally with respect to FIGS. 2 to 7 located within the chamber was assessed. Particle size measurements were conducted using a Malvern Spraytech analyzer. The location and geometry of the air inlet relative to the aerosol generating component were varied and aerosols were produced at different power levels for a range of different electrical powers. In particular, the locations and geometries depicted in FIG. 15a, FIG. 15b and FIG. 15c respectively were each assessed at different power levels. The air flow through the system was maintained for each inlet configuration. The air outlet for the system was a generally circular aperture located so as to be horizontally bisected by the generally planar aerosol generating component. The results are shown in FIG. 15d. As can be seen, when the air inlet was varied so as to bias delivery of air to a greater distance from the surface of the aerosol generating component the particle size was increased. The increase in particle size was maintained across a range of power levels. The increase in particle size was greatest for the system where the air inlet was positioned so that no air was delivered in line with the aerosol generating component (the inlet configuration of FIG. 15b). Comparing the results for the inlet configuration of FIGS. 15b and 15c shows that when more air is biased away from the surface of the aerosol generating component, the particle size can be increased.

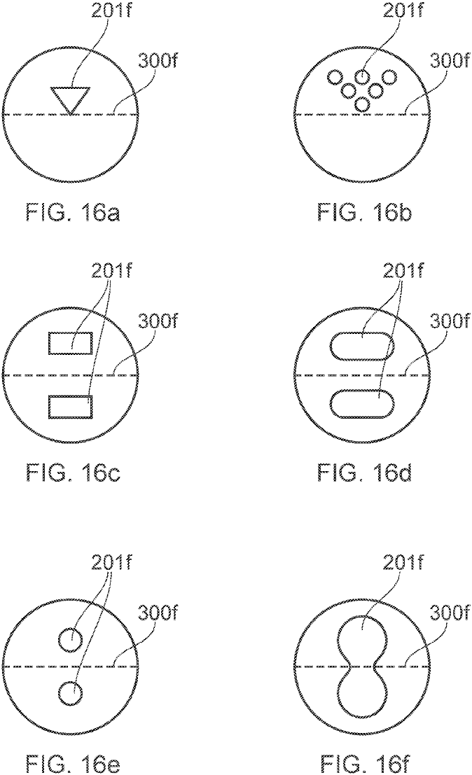

FIGS. 16a to 16f show a range of different air inlet configurations that can be adopted so as to bias the distribution of air entering the chamber away from the surface of the aerosol generating component. In each case, the aerosol generating component 300f is shown in dotted line. FIG. 16a show the air inlet 201f having a triangular aperture cross-section, with the apex of the triangle projecting towards the surface of the aerosol generating component. FIG. 16b shows the air inlet 201f being formed a plurality of circular apertures, the apertures being arranged such that a greater number are located at a greater distance from the surface of the aerosol generating component. FIGS. 16c, 16d and 16e respectively show air inlet 201f configurations of rectangular, oval and circular aperture cross-sections. FIG. 16f shows a single air inlet 201f with an “hour-glass” shape, with the neck of the “hour-glass” traversing the aerosol generating component.

The one or more air inlets of the aerosol generating chamber may span opposing sides of the generally planar aerosol generating component. Alternatively, the one or more air inlets of the aerosol generating chamber may be solely located on one side of the aerosol generating component. Such a configuration may be particularly suitable whereby vapor is released from only one surface of the aerosol generating component.

The air inlets and/or air outlets may form apertures having a cross-sectional shape selected from circular, semi-circular, triangular, square, rectangular and/ or polygonal. Exemplary aperture cross-sections may include slot, dumb-bell, hour-glass, etc. Where the air inlet and/or air outlet is formed from a single aperture, and that aperture spans opposing sides of the aerosol generating component, the cross-sectional shape is selected so as to preferentially distribute air entering the chamber away from the surface of the aerosol generating component. For example, where the aperture cross-sections is a dumb-bell, the “neck” of the dumb-bell may be horizontally bisected by the aerosol generating component.

The relative orientation between the one or more air inlets and the aerosol generating component may be fixed. Alternatively, the orientation between the one or more air inlets and the aerosol generating component may be capable of being modified by the user. This may be achieved by moving either the aerosol generating component, the one or more air inlets or both. Likewise, the geometry of the one or more air inlets may be modified so as to alter their aperture cross-section. Likewise, where there are multiple air inlets, it may be possible to modify the extent to which air enters through one or more of the inlets. For example, one or more of the air inlets may be reduced in effective cross-sectional area (or closed completely) so as to change the distribution of air entering the chamber. This has the advantage of allowing the user control over the distribution of air entering the chamber and thus the particle size of the produced aerosol.

The precise width A_{d} of the air inlet may vary as described above, but it may be that the air inlet has an aperture width of less than 1.5 mm.

The one or more air inlets may be configured such that air entering the chamber is preferentially distributed away from the surface of the aerosol generating component.

The aerosol generating chamber may comprise more than one air inlet. For example, the chamber may comprise two, three, four, five, six or more inlets. The inlets may be evenly located on respective sides of the generally planar aerosol generating component, or may be unevenly located on respective sides of the generally planar aerosol generating component. Having air inlets spanning both sides of the aerosol generating component may be advantageous where vapor is produced from each surface of the aerosol generating component and thus delivery of air to the proximity of both surfaces serves to increase the aerosol delivered by the device.

In this regard, the relative number, geometry and location of air inlets may be selected so as to alter the proportion of air being delivered to one side or other of the generally planar aerosol generating component (noting always that air entering the chamber is preferentially distributed away from the surface of the aerosol generating component). For example, a plurality of air inlets having a relatively smaller aperture cross-sectional area may provide the same overall aperture size as a single aperture having a relatively larger cross-sectional area. Where the aerosol generating chamber comprises more than one air inlet, they may be symmetrically oriented with respect to the plane of the aerosol generating component. Alternatively, where the aerosol generating chamber comprises more than one air inlet, they may be asymmetrically oriented with respect to the plane of the aerosol generating component.

The article generally comprises one air outlet. However, the chamber may comprise more than one air outlet. For example, the aerosol generating chamber may comprise two, three, four, five, six or more outlets. It may be that the configuration of the outlets matches the configuration of the inlets. Alternatively, where there are multiple inlets, there may only be one outlet (or vice versa). At least a portion of the one or more air inlets may be linearly aligned with at least a portion of the one or more air outlets. This ensures that airflow through the chamber is preferentially linear and is not diverted to any significant extent through the chamber.

According to another aspect of the present disclosure, the dimensions of the aerosol generating chamber are configured such that the velocity of air flow through the chamber is in the range of 0.05 m/s to 25 m/s. For example, the velocity may be 0.05 m/s to 20 m/s, 0.05 m/s to 15 m/s, 0.05 m/s to 10 m/s, 0.05 m/s to 5 m/s, 0.05 m/s to 3 m/s, 0.05 m/s to 2 m/s, 0.05 m/s to 1.25 m/s or 0.05 m/s to 1 m/s.

In particular, the above velocities may be imparted to the airflow through the chamber when a certain pressure drop is applied across the air inlet and air outlet of the chamber. For example, the pressure drop may be in a range of from 5 mmWG to 120 mmWG, such as from 30 mmWG to 90 mmWG, 30 mmWG to 80 mmWG, 30 mmWG to 70 mmWG, 30 mmWG to 60 mmWG, 30 mmWG to 50 mmWG, or 30 mmWG to 45 mmWG. The above velocities may be achieved at specific pressure drops, such as 30 mmWG, 35 mmWG, 40 mmWG, 45 mmWG, 50 mmWG, 55 mmWG, 60 mmWG, 65 mmWG, 70 mmWG, 75 mmWG, 80 mmWG, 85 mmWG, 90 mmWG, 95 mmWG, or 100 mmWG. It has been found that by controlling the dimensions of the aerosol generating chamber such that the above velocity range is achieved, the particle size of an aerosol entrained in the airflow can be influenced positively.

In particular, there is provided an article for use with an electrically operated non-combustible aerosol delivery system, the article comprising an aerosol generating chamber having one or more air inlets and one or more outlets defining a flow path therebetween, and a generally planar aerosol generating component suspended within the aerosol generating chamber such that the flow path is substantially parallel to the plane of the aerosol generating component, wherein respective first and second faces of the aerosol generating component project towards corresponding first and second walls of the chamber, with each wall being distanced from its respective face such that the velocity of air across each face is in the range 0.05 m/s to 25 m/s .

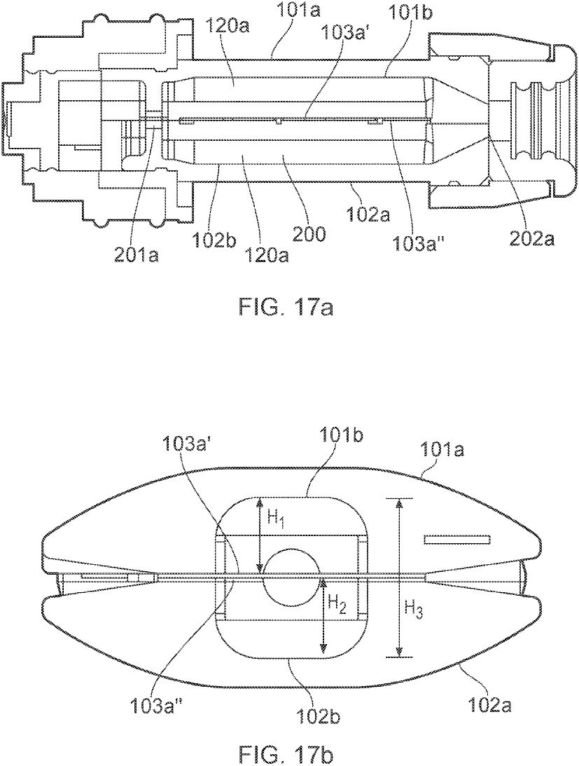

FIG. 17a shows a cross sectional view of an article of similar construction to that shown in FIGS. 2 to 6. The section is shown along the longitudinal axis of the article. FIG. 17a shows an aerosol generating component 103a held between an upper cradle 101a and a lower cradle 102a. The upper and lower cradles of FIG. 17a are slightly different to those depicted in FIGS. 2 to 6 above, but their principles of construction and liquid feeding to the aerosol generating component 103a are the same. Similarly to the cradles of FIGS. 2 to 6, a recess 120a is provided in each cradle and together these recesses form the aerosol generating chamber 200 with the aerosol generating component 103a positioned therein. An inner wall 101b of the upper cradle and inner wall 102b of the lower cradle define the recesses. The aerosol generating component 103a is comprised of an upper face 103a′ which projects towards upper cradle 101a and a lower face 103a″ which projects towards the lower cradle 102a. Air enters the aerosol generating chamber 200 via the air inlet 201a and travels substantially parallel to the upper and lower faces of the aerosol-generating component 103a.

It has been found that the velocity at which air flows through the aerosol generating chamber can influence the particle size of the aerosol produced. In particular, greater velocities have been found to be able to suppress the particle size growth of the aerosol. In some examples, the internal geometry of the aerosol generating chamber 200 is configured to provide for a velocity of air across each face of the aerosol generating component of between 0.05 m/s to 25 m/s. In order to achieve this, a pressure drop of from 5 mmWG to 120 mmWG may be applied across the air inlet and air outlet. One way of achieving this is to configure the distance between the inner walls 101b and 102b of the cradles, and the respective upper and lower faces of the aerosol generating component 103a′ and 103a″ appropriately. In this regard, FIG. 17b shows a cross-section through the chamber 200 of FIG. 17a perpendicular to the longitudinal axis of the article. The aerosol generating chamber 200 of FIGS. 17a and 17b generally has a square-cross section (noting the slightly rounded corners shown in FIG. 17b). Although other cross-sections of chambers are possible, generally the cross-section of the chamber will be generally square or rectangular. The distance between the upper face 103a′ and the upper wall 101b is shown by H₁. The distance between the lower face 103a″ and the lower wall 102b is shown by H₂. The total height between the upper and lower walls 101b, 102b is shown by H₃. It has been found that by reducing H₁ and H₂ the velocity through the chamber 200 can be increased such that particle size of the aerosol can be controlled. In some examples, H₁ and H₂ are the same. In some examples, H₁ and H₂ are different. By choosing different heights H₁ and H₂ it is possible to tailor the final particle size. H₁ and H₂ may be the same or different and have a value of less than 4 m, less than 3 m, or less than 2 mm, such as 1.9 mm, 1.8 mm, 1.7 mm, 1.6 mm, 1.5 mm, 1.4 mm, 1.3 mm, 1.2 mm, 1.1 mm or about 1 mm. H₃ may have a value of less than 8 mm, less than 7 mm, less than 6 mm, less than 5 mm, or less than 4 mm, such as 3.9 mm, 3.8 mm, 3.7 mm, 3.6 mm, 3.5 mm, 3.4 mm, 3.3 mm, 3.2 mm, 3.1 mm, 3.0 mm, 2.9 mm, 2.8 mm, 2.7 mm, 2.6 mm, 2.5 mm, 2.4 mm, 2.3 mm, 2.2 mm, 2.1 mm or about 2 mm.

It has been found that by controlling the respective distances mentioned above, it is possible to reduce the particle size of an aerosol produced by the system. In particular, for a system using the same aerosol generating component, aerosolizable material and power output, reducing each of H₁ and H₂ from 2 mm to 1 mm resulted in a significant reduction in aerosol particle size.

According to another aspect of the present disclosure, it has been found possible to also optimize the aerosol generating component. In particular, in some examples the aerosol generating component comprises a capillary structure, wherein the capillarity of a first portion of the capillary structure varies relative to the capillarity of a second portion of the capillary structure. The first portion and the second portion may have different rates of vaporization of aerosolizable material. Thus, the capillarity of different portions of the capillary structure varies in accordance with the ability of the respective portion to effect vaporization of aerosolizable material.

As has been described above, aerosol generating components can be prepared whereby some portions of the aerosol generating component are able to effect vaporization of aerosolizable material at a greater rate than other areas. This might be due to the fact that some portions of the aerosol generating component are configured to dissipate more energy during use (for example, by virtue of such portions having a higher propensity for electrical current flow). By varying the capillarity of such portions dependent on the rate at which they are able to effect vaporization of aerosolizable material, it is possible to provide that those portions having a greater rate of vaporization are able to be fed with aerosolizable material in a more optimized manner.

In this regard, it will be understood that the capillarity of a particular capillary channel is a function of the cross-sectional dimensions of that channel. Assuming the channel to have a generally circular cross-section, a relatively smaller radius will lead to a relatively greater capillarity. In some examples, the capillarity of the portions able to effect vaporization of aerosolizable material at a greater rate is greater than the capillarity of the portions able to effect vaporization of aerosolizable material at a lesser rate. Thus, where C is capillarity, in some examples Cₚₒᵣₜᵢₒₙₛ of greater rate of vaporization > Cₚₒᵣₜᵢₒₙₛ _{of} ₗₑₛₛₑᵣ ᵣₐₜₑ _{of} _{vaporization}. Therefore, those portions which vaporize aerosolizable material more quickly have a correspondingly greater capillarity. In some examples, the average pore size of the portions able to effect vaporization of aerosolizable material at a greater rate is smaller than the average pore size of the portions able to effect vaporization of aerosolizable material at a lesser rate. Thus, there is also provided an aerosol generating component having a capillary structure, wherein the aerosol generating component has portions having different average pore sizes. Thus, one or more portions of the aerosol generating component may have an average pore size in the range of 5 µm to 30 µm, and one or more other portions may have an average pore size which is different and in the range of 20 µm to 40 µm. Average pore size may be determined in this regard using a digital microscope, for example a VHX-6000 series from Keyence. For example, for each particular portion of sample being tested, the digital microscope assesses the size of each pore in the portion by distinguishing between the respective pores and fibers (using the different contrast imaged for pore and fiber). The pore sizes are then averaged over the portion being measured.

In some examples where the aerosol generating component has areas of greater and lesser rates of vaporization, the portions may have different densities. For example, the portions having a greater rate of vaporization have a density of up to 300 %, such as up to 250%, such as up to 250%, relative to the density of the portions having a lesser rate of vaporization. The difference in density is typically reflective of a difference in capillarity, with areas of greater density typically having greater capillarity. The variations in density may result from sections of the aerosol generating component being compressed relative to other sections. This compression leads to a greater density (and thus reduced average pore sizes and increased capillarity).

Accordingly, there is also provided a method for producing an aerosol generating component having a capillary structure, the method comprising the steps of providing an aerosol generating component having a capillary structure, and compressing the aerosol generating component in one or more portions to increase the density in those portions. Compressing may be carried out as is known to the skilled person, e.g. by using a stamp, roller or the like.

FIG. 11b shows an example of an aerosol generating component 103e where the capillarity of a portion of the capillary structure varies in accordance with the ability of that portion to effect vaporization of aerosolizable material. In particular, aerosol generating component 103e is formed from stainless steel fibers which have been sintered together to form a generally planar component as described generally above. The aerosol generating component 103e has slots 130 as described elsewhere herein. The aerosol generating component 103e also comprises a portion of relatively greater capillarity 140a and a portion of relatively lesser capillarity 140b. The portion of relatively greater capillarity 140a generally coincides with the portion of the aerosol generating component able to effect vaporization of aerosolizable material at a greater rate. In the example of FIG. 11b, this is achieved through the use of slots 130 directing current flow, but this could also be achieved in other ways as is described herein.

As described herein, the aerosol generating component may comprise one or more apertures which inhibit the flow of electrical current therethrough. Variations in aperture shape, size and number are described throughout this document and such variations in aperture configuration can be applied to the present aspect. Similarly, the present disclosure describes how the aerosol generating component is configured to be fed with aerosolizable material. The various variations described with respect to transferring aerosolizable material to the aerosol generating component can be equally applied to this aspect.

In some examples, the portions having greater and lesser propensity for the flow of electrical current are formed from different materials. For example, portions may be formed from different materials selected from stainless steels, such as AISI 304 or AISI 316, and heating conductor alloys, in particular NiCr alloys and CrFeAl alloys. It is also envisaged that the number/geometry of the aperture/slot features of the aerosol generating components described herein may be varied so as to influence the airflow through the aerosol generating component and/or vaporization profile of the aerosol generating component.

Thus, according to another aspect of the present disclosure, there is provided an aerosol generating component which comprises a plurality of differently sized apertures. Any of the aspects described herein may comprise an aerosol generating component with a plurality of differently sized apertures. In some examples, one or more of the apertures may be slot shaped. It may also be possible for one or more of the slot widths and/or lengths to vary. Generally, the one or more slots extend inwardly from the periphery of the aerosol generating component. Apertures or slots extending inwardly from the periphery of the aerosol generating component may reach or extend past the midpoint of the aerosol generating component. Apertures or slots may extend from opposite peripheral edges of the aerosol generating component. In this regard, it is to be understand that “apertures” or “slots” does not include surface or structural pores that may be present as an inherent part of the aerosol generating component. Rather, the terms “apertures” or “slots” mean openings which extend continuously from one surface of the generally planar aerosol generating component to the opposite surface.

The slots may take a particular form, and an exemplary slot is shown in FIG. 11b. For example, each slot may have an opening 131, a body section 132 and an apex 133. Body section 132 may be linear as shown in FIG. 11b. However, it is also possible for the body of the slot to be non-linear, or wavy. The apex (or termination as referred to above) 133 may be rounded as shown in FIG. 11b. However, other configurations are possible such as angular, oval, or droplet shaped. An advantage of having different apex configurations is that the apex configuration can be modified to as to influence the flow of electrical current in the aerosol generating component. It is also possible to have combinations of apex configurations, and/or for the apex configurations to take the same overall shape but to be oriented differently. FIG. 9d provides an example of an aerosol generating component 103g having four slots and a circular apex 133, where the width of the circular apex is enlarged relative to the width of the slot. FIG. 9e provides an example of an aerosol generating component 103g having four slots and a curved apex 133. FIG. 9f provides an example of an aerosol generating component 103g having two slots and a oval apex 133. Any combination of slot number and apex configuration is envisaged. For example, the aerosol generating component may have one, two, three, four, five or six slots. As shown in FIG. 1 1b, the opening 131 may also be flared to an extent.

In some embodiments, the number, size and shape of the apertures are distributed throughout the aerosol generating component so as to influence airflow in the aerosol generating chamber. For example, where airflow through the chamber is configured to pass through the aerosol generating component, the number, size and shape of the apertures can be selected so as to normalize airflow exiting the aerosol generating component. By “normalize” it is meant that airflow exiting the aerosol generating component is less turbulent than airflow approaching the aerosol generating component. For example, there is provided an article for use with an electrically operated non-combustible aerosol delivery system, the article comprising an aerosol generating chamber having one or more air inlets and one or more outlets defining a flow path therebetween, and a generally planar aerosol generating component suspended within the aerosol generating chamber such that the flow path is substantially transverse to the plane of the aerosol generating component, wherein the aerosol generating component comprises a plurality of differently sized apertures.

In another aspect of the present disclosure, it is envisaged that a temperature profile within the aerosol generating chamber varies from the one or more air inlets to the one or more air outlets. In particular, it is envisaged that during activation of the aerosol generating component a first temperature profile having a negative gradient is established along a portion of the flow path from inlet to outlet. It has been surprisingly found that when such a temperature profile having a negative gradient is established, particle size growth of the aerosol can be suppressed. Therefore, by controlling the gradient of the temperature profile, it is possible to control the particle size of the aerosol, which can be positive from a sensorial aspect.

Accordingly, there is provided an article for use with an electrically operated non-combustible aerosol delivery system, the article comprising a generally planar aerosol generating component suspended within an aerosol generating chamber, the chamber having one or more air inlets and one or more outlets defining a flow path therebetween, wherein during activation of the aerosol generating component a first temperature profile having a negative gradient is established along a portion of the flow path from inlet to outlet.

By “temperature profile with a negative gradient” it is meant that a temperature at an upstream location of the aerosol generating chamber is greater than a temperature at an corresponding downstream location of the aerosol generating chamber. Thus, the temperature is generally higher at an upstream end of the chamber than at a downstream end.

Typically, the aerosol generating component defines a longitudinal axis which extends along the flow path. In some examples, the negative temperature gradient is established along less than 50% of the flow path. In some examples, the negative temperature gradient is established along less than 20% of the flow path. In some examples, the negative temperature gradient is established along less than 5% of the flow path. In this regard, for a given starting temperature, limiting the extent to which the negative temperature gradient extends influences the rate at which the temperature drops within the chamber.

In some examples, the peak temperature is established in proximity to the one or more air inlet(s) of the aerosol generating chamber. For example, the peak temperature may be established within 20%, 15%, 10% or 5% from the opening of the air inlet(s) into the aerosol generating chamber. In this regard, the % proximity is based upon a linear pathway from the air inlet to the air outlet of the aerosol generating chamber.

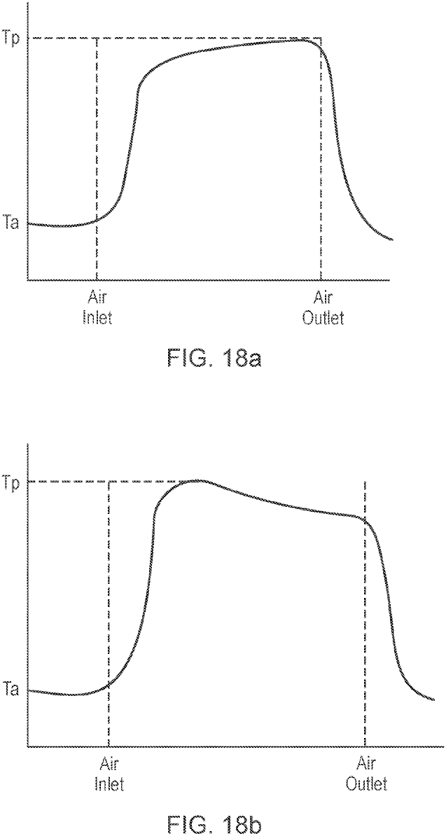

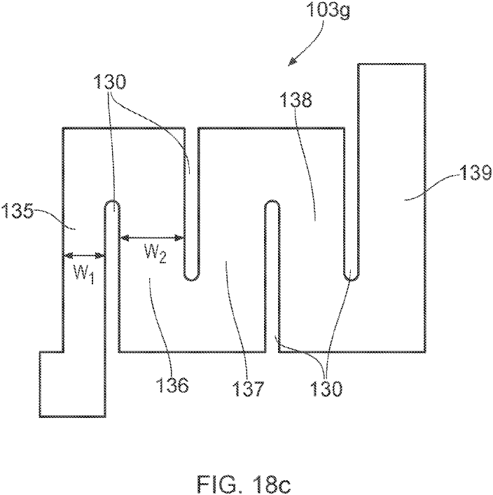

Establishing a temperature profile with a negative gradient may be achieved in a number of ways. For example, it may be that an additional heater is located in proximity to the one or more air inlets such that incoming air is subjected to heating. As the airflow moves through the aerosol generating chamber it is subjected to relatively less heating than at an upstream location and subsequently cools thus establishing the negative temperature gradient. Other ways of establishing such a gradient will be apparent to the skilled person. It should be noted in this regard that under normal circumstances in prior art devices the airflow that has travelled past the heater will begin to cool and thus a negative temperature gradient will be established at some point in time over the total flow path. However, in the system described herein, the aerosol generating component is generally disposed parallel to the airflow through the device. This has the effect that incoming air is continually heated as it travels the length of the aerosol generating component. This is illustrated in FIG. 18a, where the Y axis represents the temperature of the airflow as it travels a distance through the aerosol generating chamber, represented by the X-axis. Tₐ represents the temperature of the incoming “ambient” air, and T_{P} represents the peak temperature within the aerosol generating chamber of the device. It will be observed that the temperature of the airflow rises rapidly on entering the aerosol generating chamber and then continues to rise up to T_{P} due to the cumulative heating it experiences. The temperature of the air then drops rapidly upon exiting the aerosol generating chamber. FIG. 18b illustrates the temperature profile of an aerosol generating chamber configured according to the present aspect of the disclosure. Similarly to in FIG. 18a, the temperature rises rapidly from Tₐ upon entry into the aerosol generating chamber. However, contrary to in FIG. 18a, according to the present aspect T_{P} is established at an upstream location of the aerosol generating chamber. The temperature then decays along the chamber until the same rapid drop in temperature is experienced upon exit.

Other ways of establishing a negative temperature gradient within the chamber are also possible. For example, the aerosol generating component may be configured to dissipate greater energy, i.e. heat, at an upstream location relative to a downstream location. This might be achieved in a number of ways. For example, the aerosol generating component may be configured with a capillary structure at an upstream portion which has a capillarity inferior in terms of feed rate compared to a capillary structure at a corresponding downstream portion. This will result in aerosolizable material being fed less rapidly to the upstream portion. Since the presence of aerosolizable material effectively acts to moderate the temperature of the aerosol generating component during heating, where less aerosolizable material is being provided there will be a greater localized temperature. Alternatively, a similar effect can be achieved by configuring the aerosol generating component such that an upstream portion experiences relatively greater resistive heating than a corresponding downstream portion. In some examples, this is achieved by configuring an upstream portion of the aerosol generating component such that said portion has a higher resistance than a corresponding downstream portion. This higher resistance can, for example, be imparted to the portion by forming said portion of a material with a higher electrical resistance or by modifying the geometry of said portion. An example of the resistance being modified by modifying the geometry is shown in FIG. 18c. FIG. 18c shows an aerosol generating component 103g generally similar to the aerosol generating component 103 described above with respect to FIGS. 2 to 7. Thus, aerosol generating component 103g is in this embodiment formed from stainless steel fibers which have been sintered together to form a generally planar aerosol generating component with a capillary structure. Aerosol generating component 103g has slots 130 as also described elsewhere. However, aerosol generating component 103g has an upstream portion 135 which has an electrical resistance which is higher than that of a subsequent downstream portion 136. In this regard, although portions 135 and 136 are formed from the same material and have the same general structure, portion 135 has a width W₁ which is smaller than the width W₂ of portion 136. In general, the width of the upstream portion will be constant, but in some examples, the width of the upstream portions varies. For example, the width may vary due to the slot body being angular thus forming a somewhat tapered profile. It is also possible that the width of an upstream portion is reduced relative to other portions of the aerosol generating component as a result of a deviation in the perimeter of the aerosol generating component at the relevant portion. Thus, it may be that a notch or other indentation into the aerosol generating component restricts the width of the upstream portion. For example, FIG. 9d to FIG. 9f show examples of the aerosol generating components where a notch 137 is present on the most upstream portion of the aerosol generating component (where the direction of flow is from left to right). The notch serves to restrict current flow through the upstream portion and therefore leads to an increase in resistance and thus heat generation. It will be appreciated that any form of notch, cut-out, etc., can be formed in this portion provided that it is sufficient to result in an increase in the electrical resistance of the portion compared to the next downstream portion. In FIGS. 9d and 9f, notches 137 have a curved profile. In FIG. 9e, the notch has a somewhat liner profile. The dimensions and/or number of notches/cut-outs can be varied so as to achieve the desired electrical resistance of the upstream portion. It will also be noted that in FIGS. 9d to 9f there is a corresponding notch/cut-out at the downstream most portion of the aerosol generating component. This additional notch is provided so as to allow for ease of manufacturing. More particularly, by providing a notch/cut-out at the respective most upstream and most downstream portions of the component, it is possible to prepare an aerosol generating component that has a degree of rotational symmetry. This means that it is easier to orientate the aerosol generating component correctly during manufacture. Accordingly, in a further aspect, there is provided an aerosol generating component having a degree of rotational symmetry. The degree of rotational symmetry may be 2-fold. The aerosol generating component may be substantially flat or planar and the degree of rotational symmetry is with respect to the plane of the aerosol generating component. The aerosol generating component may comprise one or more apertures, such as slots as described herein. The slots may terminate at an apex. The apex may be take a variety of profiles as described herein. The aerosol generating component may be configured such that the electrical resistance of the component varies between an upstream portion and a downstream portion as described herein.

In the specific example of FIG. 18c, portion 135 has a constant width of 1.3 mm and portion 136 has a constant width of 2.0 mm. Thus, in some embodiments the ration of W₁ to W₂ less than 1, such as less than 0.9, less than 0.8, less than 0.7, or less than 0.6. Limits on the ratio between portions may mean that a lower limit of the ratio may be about 0.5. As a result of this ratio, electrical current passing through portion 135 experiences a relatively greater resistance compared to when it travels through portion 136. Since P = I²R, the greater resistance experienced through portion 135 leads to greater power being distributed to portion 135. This greater power leads to a relatively increased temperature locally in portion 135. Moreover, this is exacerbated due to the relatively reduced amount of material in portion 135 compared to portion 136, and thus there is less material in 135 to distribute the relatively increased power. Such a result leads to portion 135 being heated to a temperature higher than portion 136. Since further downstream portions 136, 138 and 139 are dimensioned similarly to portion 135, they will be heated to a similar extent as portion 136. Thus, a negative temperature gradient is established from an upstream location to a downstream location. Accordingly, in some examples, the aerosol generating component has an upstream portion having a relatively greater electrical resistance than a subsequent downstream portion. In some examples, respective portions of the aerosol generating component may be defined by an opening extending from the perimeter of the aerosol generating component. The opening may be a slot which extends generally perpendicularly to the longitudinal axis of the aerosol generating component. As described above, the width of an upstream portion may be less than the width of a downstream portion. In some examples, the aerosol generating component comprises two, three, four, five or six portions. At least one of the upstream portions may have a width less than the width of a downstream portion. Alternatively, two or more of the upstream portions may each have a width less than the width of a portion downstream (of each of the upstream portions).

In some examples, the temperature gradient may be divided into two or more segments having different gradients of temperature change. Each segment may comprise one or more upstream/downstream portions as described above in the context of portions 135 to 139. Thus, a second or subsequent temperature profile may be established along a subsequent portion of the flow path. The second or subsequent temperature profile may have a positive, neutral or negative temperature gradient. Where it has a negative gradient, it may be smaller than the first temperature profile. Alternatively, the second or subsequent temperature profile may have a negative gradient which is greater than the first temperature profile. The second or subsequent temperature profile may extend for the remainder of the flow path to the air outlet.

In line with the above aspect, there is also envisaged an aerosol generating component for use with an electrically operated non-combustible aerosol delivery system, wherein the aerosol generating component defines a longitudinal-axis and is configured to be heated heterogeneously along its longitudinal axis. For example, during activation of the aerosol generating component a first temperature profile with a negative gradient is established along a portion of the longitudinal axis of the aerosol generating component. The negative gradient may be established along less than 50%, less than 20%, less than 15%, less than 10% or less than 5% of its longitudinal axis. Heterogeneous heating may be imparted to the aerosol generating component as described above, e.g. by varying the feeding of aerosolizable material to portions of the aerosol generating component, or by altering the flow of current through upstream/downstream portions/segments of the aerosol generating component as described above.

Due to the upstream/downstream configuration of the above-mentioned portions 135 to 139, it is clear that the impact on temperature within the aerosol generating chamber will also take a corresponding upstream/downstream configuration. However, as described above in connection with the broader disclosure, the aerosol generating component may be one having portions of greater and lesser rates of vaporization configured in other ways which generally result in a lateral temperature gradient being established. Any of the embodiments described above with respect to the modulation of rates of vaporization of the aerosol generating component may be employed in the context of the present aspect.

The articles described herein may further comprise a mouthpiece which is in fluid communication with the one or more air outlets of the aerosol generating chamber.

The articles described herein may comprise an outer housing within which the aerosol generating chamber and aerosol generating component are located. However, it may be that the article is composed of just the aerosol generating chamber and aerosol generating component. Where such an outer housing is present, it may further accommodate the store for aerosolizable material mentioned above. Such a housing may also accommodate the mouthpiece and any connectors required to ensure connection with an electrically operated aerosol delivery device (discussed further below). The outer housing may surround the aerosol generating chamber so as to form the above mentioned store for aerosolizable material.

The articles described herein are typically for use with an electrically operated aerosol delivery system. For example, the electrically operated aerosol delivery system may comprise the article described herein and an electrically operated aerosol delivery device. The article and the device may be connected so as to form the electrically operated aerosol delivery system. In this regard, the electrically operated aerosol delivery device generally comprises a power source and a controller. Both the article and the electrically operated delivery device may comprise electrical connectors which mate with each other so as to facilitate current flow to the article. Alternatively, the device may include an inductor coil which is used to generate an alternating magnetic field which can be used to induce electrical current flow within the aerosol generating component of the article. The controller of the device operates to direct power from the power source to the article when instructed to do so by a user. For example, the device may include some form of user interface, e.g. a touch sensor, button or the like, which may be operated by the user when an aerosol is to be generated. Alternatively, or additionally, the controller may receive a signal from one or more sensors (located either on the device or article) following the detection that an aerosol is to be produced. In response to such a signal, the controller operates to direct power from the power source to the article. Such sensors include air flow sensors, pressure sensors etc.

Upon connection of the article and the electrically operated aerosol delivery device, an airflow path may be created which facilitates the passage of air from the external environment to the one or more air inlets described herein, through the aerosol generating chamber, through the one or more air outlets and through a mouthpiece of the article.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An article for use with an electrically operated non-combustible aerosol delivery system, the article comprising:
a generally planar aerosol generating component suspended within an aerosol generating chamber, the aerosol generating chamber having one or more air inlets and one or more outlets defining a flow path therebetween, wherein during activation of the aerosol generating component a first temperature profile having a negative gradient is established along a portion of the flow path from the one or more air inlets to the one or more outlets.

2. The article according to claim 1, wherein a longitudinal axis of the aerosol generating component is substantially parallel to the flow path.

3. The article according to claim 1, wherein the negative temperature gradient is established along less than 50% of the flow path.

4. The article according to claim 3, wherein the negative temperature gradient is established along less than 20% of the flow path.

5. The article according to claim 1, wherein the negative temperature gradient is established along a first 5% of the flow path.

6. The article according to claim 1, wherein the negative temperature gradient is established from a peak temperature of above 250° C.

7. The article according to claim 6, wherein the peak temperature is established in proximity to the one or more air inlet(s) of the aerosol-generating chamber.

8. The article according to claim 7, wherein the peak temperature is established within 20% of an opening of the one or more air inlets into the aerosol generating chamber.

9. The article according to claim 1, wherein the aerosol generating component is configured to dissipate greater energy at an upstream location relative to a downstream location.

10. The article according to claim 9, wherein the aerosol generating component is configured with a capillary structure at an upstream portion which has a capillarity inferior in terms of feed rate compared to a capillary structure at a corresponding downstream portion.

11-28. (canceled)

29. The article according to claim 9, wherein an upstream portion of the aerosol generating component is configured to have a relatively greater electrical resistance than a corresponding downstream portion.

30. The article according to claim 1, wherein the aerosol generating component further comprises one or more apertures or slots extending from one surface of the planar aerosol generating component to an opposite surface.

31. The article according to claim 30, wherein the one or more apertures or slots originate from a periphery of the aerosol generating component.

32. The article according to claim 31, wherein the one or more apertures or slots are arranged perpendicularly to a longitudinal extent of the aerosol generating component such that a plurality of portions extending perpendicularly to the longitudinal extent of the aerosol generating component are formed.

33. The article according to claim 32, wherein a width of an upstream portion is less than a width of a subsequent downstream portion.

34. The article according to claim 33, wherein a ratio of the width of the upstream portion to the subsequent downstream portion is less than 1, but not less than 0.5.

35. The article according to claim 1, wherein a second temperature profile is established along a subsequent portion of the flow path.

36. The article according to claim 35, wherein the second temperature profile has a negative gradient which is smaller than the first temperature profile.

37. The article according to claim 35, wherein the second temperature profile has a negative gradient which is greater than the first temperature profile.

38. An aerosol generating component for use with an electrically operated non-combustible aerosol delivery system, the aerosol generating component defining a longitudinal axis and being configured to be heated heterogeneously along the longitudinal axis.

39. The aerosol generating component according to claim 38, wherein during activation of the aerosol generating component a first temperature profile with a negative gradient is established along a portion of the longitudinal axis of the aerosol generating component.

40. The aerosol generating component according to claim 39, wherein the negative gradient is established along less than 50% of the longitudinal axis of the aerosol generating component.

41. The aerosol generating component according to claim 40, wherein the negative gradient is established along less than 20% of the longitudinal axis of the aerosol generating component.

42. The aerosol generating component according to claim 39, wherein the negative gradient is established along a first 5% of the longitudinal axis of the aerosol generating component.

43. The aerosol generating component according to claim 38, wherein the aerosol generating component is formed from a woven or weave structure, a mesh structure, a fabric structure, an open-pored fiber structure, an open-pored sintered structure, an open-pored foam, or an open-pored deposition structure.

44. A non-combustible aerosol provision system comprising the article of claim 1, and a device comprising a power source and a control unit.

45. A non-combustible aerosol provision system, comprising the aerosol generating component of claim 38, and a device comprising a power source and a control unit.
